(19) United States
(12) Patent Application Publication
(10) Pub. No.: US 2013/0149297 A1
(43) Pub. Date: Jun. 13, 2013

(54) ROLE OF FRAGILE X MENTAL RETARDATION GENE AND PROTEIN IN CANCER METASTASIS

(71) Applicant: Claudia Bagni, Heverlee (BE)

(72) Inventor: Claudia Bagni, Heverlee (BE)

(73) Assignees: VIB VZW; Katholieke Universitelt Leuven K.U. Leuven R&D

(21) Appl. No.: 13/807,766

(22) PCT Filed: Jul. 4, 2011
(86) PCT No.: PCT/EP2011/061240

§ 371 Date: Feb. 21, 2013

Related U.S. Application Data
(60) Provisional application No. 61/398,884, filed on Jul. 2, 2010.

(51) Int. Cl. C07K 16/18; C12N 15/113

(52) U.S. Cl. CPC C07K 16/18; C12N 15/113
USPC 424/130.1; 514/44 A; 536/24.5; 530/389.1; 435/6.14

(57) ABSTRACT

The present application relates to the field of cancer. Surprisingly, it was shown that the Fragile X mental retardation gene (Fmr1) products such as the FMRP protein, primarily implicated in mental retardation, are upregulated in metastasizing tumours. It is shown how Fmr1 gene products can be used as a marker for cancer metastasis and how inhibition of these gene products may help prevent or reduce metastasis.

### FIELD OF THE INVENTION

The present application relates to the field of cancer. Surprisingly, it was shown that the Fragile X mental retardation gene (Fmr1) products such as the FMRP protein, primarily implicated in mental retardation, are upregulated in metastasizing tumours. It is shown how Fmr1 gene products can be used as a marker for cancer metastasis and how inhibition of these gene products may help prevent or reduce metastasis.

### BACKGROUND

The Fragile X mental retardation protein (FMRP) is an RNA binding protein involved in multiple steps of RNA metabolism in neurons. FMRP is lacking or mutated in patients with the Fragile X syndrome (FXS), the most common form of inherited mental retardation in human with an incidence of 1:2000 in males and 1:4000 in females (Chiurazzi et al., 2003; Bassell and Warren, 2008). In brain FMRP regulates mRNA stability, localization and translation of some key mRNAs involved in cytoskeleton and spine remodeling (De Rubeis and Bagni, 2009). Absence of FMRP leads to spine dysmorphogenesis possibly due to an impaired cytoskeleton formation and receptor mobility at synapses (Bagni and Greenough, 2005). Cell migration, axonal and dendritic projection, and synaptic targeting and pruning are key processes that, surprisingly, share common regulatory pathways with cancer progression and metastatization i.e. cytoskeleton remodeling and cell adhesion (Schmid and Maness, 2008; Kim et al., 2009). Interestingly, individuals with Fragile X Syndrome have been reported to have a decreased risk of cancer (Schultz-Pedersen et al., 2001) and one patient showed unusual long survival with a glioblastoma (Kalkunte et al., 2007). On the other hand, instability of FMR1 CGG repeat sequences has also been observed in hereditary nonpolyposis colon cancer (Fulchignoni-Lataud et al., 1997), so there is no established link between the FMR1 gene products and cancer.

Cancer is the term typically used to refer to a class of diseases in which a group of cells display uncontrolled growth (division beyond the normal limits), invasion (intrusion on and destruction of adjacent tissues), and sometimes metastasis (spread to other locations in the body via lymph or blood). Metastasis is a highly regulated, multistep process in which cancerous cells shed from the primary tumor and enter the circulatory system, where they interact extensively with host cells before they colonize the target organ (Sahai, 2007; Chaffer and Weinberg, 2011). Some of these steps involve cell-cell interactions, and molecules involved in cell adhesion structures are crucial for tumor cell dissemination and metastasis formation (Olson and Sahai, 2009; Schmalhofer et al., 2009). One of the hallmarks of an aggressive tumor is its propensity to metastasize, and the understanding of this process is of high importance for the treatment of cancer. The ability of cancer cells to disseminate from primary tumors and form metastasis is highly dependent on tumor cell motility, intravasation, transit in the blood or lymph, extravasation and growth at a new site (Hood and Cheresh, 2002; Sahai, 2007). Remodeling of the cytoskeleton and changes of cell surface are crucial for tumor cell migration and metastasis (Olson and Sahai, 2009). The same goes for loss of cell adhesion, a typical feature observed in epithelial-mesenchymal transition (EMT). A loss of epithelial-cell markers and gain of mesenchymal-cell markers has been observed in patient tumor samples, particularly at the leading edge or invasive front of solid tumors such as non-small cell (NSCLC), pancreatic, colorectal, and hepatocellular cancers. Such changes in phenotypic epithelial-like and mesenchymal-like cellular markers have been associated with the degree of tumor progression. The loss of epithelial-cell markers (e.g. E-cadherin, gamma catenin, others) is associated with disease progression and metastatic potential of a tumor. It has become evident that cancer cells can dedifferentiate through activation of specific biological pathways associated with EMT, thereby gaining the ability to migrate and invade. Hence, what has been observed experimentally regarding EMT and normal organ development is also thought to apply in the progression of solid tumors (Micalizzi and Ford, 2009; Thiery et al., 2009): transcriptional reprogramming processes whereby epithelial tumor cells lose cell polarity and cell-junction proteins and at the same time acquire protein mesenchymal-cell markers (e.g. vimentin, fibronectin, others) and signal transduction activities associated with mesenchymal cells facilitating migration and survival in an anchorage-independent environment, and ultimately metastasis at distal sites.

Increasing evidence suggests that EMT (and the reverse MET) are central regulators of cellular plasticity in carcinomas and have important roles in therapeutic resistance, tumour recurrence and metastatic progression (Polyak and Weinberg, 2009). Owing to the clinical importance of the EMT-induced processes, inhibition of EMT is an attractive therapeutic approach that could have significant effect on disease outcome. However, the complexity of the signalling networks that regulate induction of EMTs pose significant challenges. Despite the large amount of data accumulated on the molecular mechanisms underlying EMT and MET and the undoubted importance of these processes in cancer, no truly useful clinical results have been obtained to date. As metastasizing cancer is one of the most important causes of death worldwide, it would be advantageous to have new methods of diagnosing the metastatic potential of tumours, as well as further options of treating metastasizing cancers.

### SUMMARY

As mentioned above, few reports showed a decreased risk of cancer and neuroprotection in individuals with Fragile X (Schultz-Pedersen et al., 2001; Kalkunte et al., 2007). As it is unknown whether this is a causative effect or a correlation, and if there is a causative link whether this is direct or indirect, the possible effect of FMRP absence in tumor generation and cancer progression was investigated. It is shown here that FMRP expression is deregulated in different human carcinomas and that, in primary breast tumors, high FMRP levels correlate with prognostic indicators of aggressive breast cancer and metastasis. Additionally, FMRP is highly expressed in the invasive front of metastatic tumors as well as in distal metastases. Furthermore, it is demonstrated that reduction of FMRP in tumor cells decreases their ability to form lung metastases after orthotopic and vein tail injections in wild-type mice. These data allow us to propose, for the first time, a role of FMRP in the metastatic phenotype of breast cancer. Interestingly, FMRP is inversely correlated with E-cadherin expression, a key adhesion molecule involved in epithelial to mesenchimal transition. This has consequences for both diagnosis and treatment of cancer, especially metastasizing cancer.

Thus, according to a first aspect, methods of assessing metastatic potential of a tumour in a subject are provided, which involve determining the levels of FMR1 gene product in said tumour. In other words, FMR1 gene product is provided for use in a method of diagnosing metastatic potential of tumours. According to particular embodiments, the levels of FMR1 gene product are measured at the invasive front of the solid tumor, i.e. at the tumour-host interface. If levels of FMR1 gene product are increased when compared to control, this is indicative of an increased risk of metastasis and thus of a more aggressive tumor. Relevant controls may be selected by the skilled person and include for instance non-tumorous tissue of the organ where the tumour is located. Thus, according to specific embodiments, the methods further entail the correlating of increased levels of FMR1 gene product to increased risk of metastasis, and/or vice versa: decreased or absent levels of FMR1 gene product to low(er) risk of metastasis.

According to other very particular embodiments, the FMR1 gene product levels of lymph node negative tumours can be correlated to an increased risk for metastasis of these tumours (see e.g. FIGS. 1B and 1C).

According to particular embodiments, the FMR1 gene product whose levels are determined is Fmr1 mRNA. This can be the total of all mRNA isoforms, or one or more specific mRNAs may be determined. Examples include, but are not limited to, ISO1 (the longest transcript encoding the longest protein), ISO6 (lacking an alternate segment and using a different splice site in the 3′ coding region which shifts the reading frame, compared to variant ISO1), ISO7 (lacking an alternate segment, compared to variant ISO1), ISO9 (lacking an alternate segment and using a different splice site in the 3′ coding region, compared to variant ISO1), and ISO12 (lacking two alternate segments and using a different splice site which changes the reading frame, compared to variant ISO1).

According to alternative particular embodiment, the FMR1 gene product whose levels are determined is FMRP protein. The same considerations regarding isoforms apply: most particularly, all FMRP protein isoforms may be detected (e.g. using an antibody against a common epitope), although it is envisaged as well that only some specific isoform(s) will be detected (e.g. using an antibody against the different C-termini). Examples of antibodies generated against the C-terminus include those of Ferrari et al. (Ferrari et al., 2007) and Brown et al. (Brown et al., 2001). An example of an antibody against the N-terminus of FMRP is the commercially available 1C3 antibody (Chemicon).

Of note, it is envisaged as well that both FMR1 mRNA and FMRP protein are determined. In this case, the isoforms to be detected can be all isoforms for both mRNA and protein, identical isoforms (wholly overlapping), or different isoforms (partly or not overlapping), depending on the setup of the experiment. With identical isoforms, it is meant that the mRNA isoform encodes for the corresponding protein isoform.

The tumour of which the FMR1 gene product levels are determined can be any tumour, particularly any tumour at risk of metastasis. It is particularly envisaged that the tumour is selected from the group of breast cancer, colon cancer, and bladder cancer. According to most specific embodiments, the tumour of which the FMR1 gene product levels are determined is breast cancer. According to even more specific embodiments, the breast cancer is lymph node negative breast cancer. Lymph node status can be determined prior to, concomitant with or after determination of the FMR1 gene product levels, either independently or as part of the same diagnosis process.

According to particular embodiments, determining the FMR1 gene product levels occurs in vitro, e.g. on a tumour sample or biopsy.

According to a further aspect, FMR1 inhibitors are provided for use as a medicament. Indeed, to our knowledge, this is the first time it is shown that FMR1 gene product inhibition can be beneficial, since loss of FMR1 function usually is associated with adverse effects, as in Fragile X syndrome. According to further particular embodiments, FMR1 inhibitors (or, for that matter, pharmaceutical compositions comprising FMR1 inhibitors) are provided for use in preventing and/or treating metastasis of a tumour in a subject. In other words, methods of preventing and/or treating metastasis of a tumour in a subject are provided, comprising administering an FMR1 inhibitor to the subject. Slowing the progress of further metastasis or decreasing the number of additional metastasis when compared with a non-treated control is also envisaged under the term preventing and/or treating.

A FMR1 inhibitor is any inhibitor of functionally active FMR1. According to particular embodiments, the FMR1 inhibitor inhibits the FMR1 function at the nucleic acid level. Nucleic acid level inhibition can occur in the nucleus and/or the cytoplasm. This can be at DNA level (e.g. using gene inactivation, for instance via zinc finger nucleases or gene therapy. Note that inhibition should not take place in the embryo or during early development, in order not to interfere with neural development, which otherwise may result in Fragile X syndrome), or according to particular embodiments, through interfering with Fmr1 mRNA. This may for instance be achieved through the use of Fmr1 siRNA. Complete ablation of functionally active Fmr1 mRNA is not necessary to achieve therapeutic benefit: decreasing the levels of functional Fmr1 to a sufficient extent (e.g. 50%) is already beneficial. mRNA may also be inhibited by affecting its stability (as also happens in Fragile X with the expanded trinucleotide repeats), e.g. via the use of RNA binding proteins (RBPs) and microRNAs (miRs), or any combination thereof.

According to alternative particular embodiments, the inhibitor of FMR1 function inhibits at the protein level, thus by inhibiting FMRP function. This may be achieved via anti-FMRP antibodies or antibody variants (single chain antibodies, scFv, Fc fusion proteins), nanobodies, inhibitory peptides and the like.

Analogous to the methods for diagnosis, the tumour which is treated for prevention and/or treatment of metastasis by inhibiting FMR1 gene product function can be any tumour, particularly any tumour at risk of metastasis. It is particularly envisaged that the tumour is selected from the group of breast cancer, colon cancer, bladder cancer and stomach cancer. According to most specific embodiments, the tumour of which the FMR1 gene product function is inhibited is breast cancer. According to further specific embodiments, the breast cancer is lymph node negative breast cancer.

Of note, the embodiments described herein are not mutually exclusive and thus may be combined with each other, unless explicitly stated otherwise.

### BRIEF DESCRIPTION OF THE FIGURES

FIG. 1. FMRP and Fmr1 mRNA are highly expressed in human breast cancer and distant metastasis.

(A) Expression analysis of FMRP on human multi-tumor TMA. FMRP positive samples (n), number of tumors analyzed for normal (N) and tumor tissue (T) and the percentage of FMRP positive (%) are shown. Statistical analysis was performed by Contingency Table analysis with Pearson chi-square test (JMP™ IN 5.1). (B) FMRP expression in breast cancer tissues. FMRP protein expression was analysed on a subset of previously generated breast cancer tissue microarrays (Confalonieri et al., 2009). The association between the clinical-pathological variables of the tumors and FMRP expression was evaluated by Fisher's exact test. *Not all clinical parameters were available for the entire cohort. FMRP-IHC, FMRP expression by immunohistochemical analysis; pT, primary tumor stage; nodal status, lymph nodes involvement; Grade, tumors were graded according to Elston and Ellis, 1991; ER, estrogen receptor status; PgR, progesterone receptor status; Ki-67, proliferation index; ErbB2 or HER2/neu, Human Epidermal growth factor Receptor 2 status. (C) FMR1 mRNA expression analysis on four different breast cancer datasets. Upper panel, the TRANSBIG (Desmedt et al., 2007), EMC-344 (Wang et al., 2005), MSK-99 (Minn et al., 2005), NKI-295 (van de Vijver et al., 2002) cohorts revealed an increased expression of FMR1 mRNA (expressed as a percentage) in metastatic tumors to distal organs (i.e. TRANSBIG) or specifically to lungs (i.e. EMC-344, MSK-99 and NKI-295) compared to non metastatic tumors. pts (n), number of patients. Significance of FMR1 mRNA increased expression is calculated using Welch's t-test. NC (Not Changed). (D) Kaplan-Meier curve showing the probability of having metastasis to lung (low FMRP levels in red (top line and numbers) and high levels in blue (lower line and numbers in graphs)). n=number of patients; events=number of patients with lung. Left curve: NKI-295 node-negative dataset (n=151), middle curve: NKI-295 node-positive dataset (n=144), right curve: all dataset metastatic to the lung (pts=639 excluding TRANSBIG), P values were calculated using the Log-rank test. (E) FMRP differential expression in primary breast tumors and matched distal metastases. Representative images of FMRP are shown on the left, the average anti-FMRP IHC score of the cohort on the right. **, P<0.01, Student's ttest. Scale bars are 50 μm.

FIG. 2. Engineering of the TMAs used for the screening analysis of FMRP Expression and specificity of antibodies used.

(A) TMA combinations used for the screening of FMRP expression. Four different TMAs were engineered. The number of cases, for each type of tumors and matched controls (whenever available), deposited on individual TMAs is reported. In each column, the first number refers to the number of tumor cases and the second to the number of normal matched samples (T/N). As indicated, normal counterparts were not always available. Each case was deposited in duplicate. In colorectal, lung, larynx, prostate and bladder tumors, normal samples were derived from the same patients whenever possible. In breast tumors, the normal samples were fibroadenomas (indicated by asterisks) and only a couple of cases were paired normal glands. (B) Expression analysis of FMRP on human multi-tumor tissue microarrays. The number of FMRP positive samples (positive n), total number of analyzed tumours (in parenthesis), for normal (N) and tumour tissue (T) for the cancer type as well as the percentage of FMRP positive (positive %) are shown.

For the normal tissue counterparts we specifically considered: for lung, normal epithelial cells of both alveoli and bronchi; for larynx, normal ciliated and squamous epithelial cells; for prostate, glandular epithelium. In normal lung tissue, bronchial epithelial cells were more positive (only 3 cores arrayed on the TMAs displayed bronchial epithelial cells, two of which resulted positive) than alveolar epithelial cells (all negative for FMRP expression). (C) Western blot analysis of FMRP expression in WT and Fmr1 KO mouse brain using specific FMRP antibodies (Ferrari et al., 2007). The signal appears clear in WT extracts and absent in Fmr1 KO extracts, showing specificity.

FIG. 3. Cancer and FXS Registry in England.

Eight cancers were identified amongst the 226 women from the Central Manchester Foundation Trust, England: premutation, affected and unaffected full mutation alleles. When premutation and affected full mutation female carriers were analyzed, 5 cancer cases were observed compared to an expected of 13.96, giving a P value for a deficit of cancers based on a Poisson distribution, a one sided test of 0.006. Only 1 case of breast cancer was detected compared to an expected of 5.14, giving a P value for a deficit of breast cancer based on a Poisson distribution, a one sided test of 0.04. The described analysis was performed comparing normal and FXS population from 0-79 years old.

FIG. 4. Clinical and pathological information of the consecutive cohort of breast cancer patients.

(A) Patients from IEO were classified as follows: The clinical and pathological information of the consecutive cohort of breast cancer patients is reported. pT, primary tumor stage; Nodal status, Lymph nodes involvement; †Grade, tumors were graded according to the system of Elston and Ellis(9) where G1 indicates well differentiated, G2 moderately differentiated, and G3 poorly differentiated; ER, estrogen receptor status; PgR, progesterone receptor status; Ki67, proliferation index; ErbB2 or HER2/neu, Human Epidermal growth factor Receptor 2 status; all events, loco-regional relapse, distant metastasis or controlateral breast cancer; distant events, distant metastasis. Disease recurrence (all events and distant events) was within 5 years (Veronesi et al., 2003). IHC-FMRP, FMRP expression measured by IHC was considered negative when IHCFMRP score ≦1.0 and positive when >1.0. (B) Patients from University Hospital Leuven (Belgium) were classified as above. Disease recurrence (all events and distant events) was within 5-10 years. (C) Patients from Hospital Casa Sollievo (Italy) were classified as above and patients had a follow up of 2 years (Barbano et al., 2011). *Not all the parameters were available for all the patients analysed.

FIG. 5. FMRP expression in normal and breast tumor mouse tissues.

FMRP levels in normal and primary tumor tissues by Western blotting analysis. Lanes 1-3, protein extracts from normal breast tissues (mammary fat pad); lanes 4-11, 8 different murine breast tumors. FMRP levels were analysed by Western blot using specific FMRP antibodies (Ferrari et al., 2007) (left upper panel), the signal was normalised for Coomassie staining of the membrane (left lower panel). The right panel reports the quantification of the band intensities. P<0.01, Student's t test.

FIG. 6. FMRP levels influence cell-cell adhesion and metastasis formation.

(A) Number of lung metastasis per tumor weight, 29 and 35 days after orthotopic injection of control 4T1 and TS/A cells respectively (P<0.001, Student's t test). (B) As in panel (A), using 4T1 cells transduced with control or anti-FMRP shRNA viruses (n=13, P<0.05, Student's t-test). (C) As in panel (A) with control and Fmr1-silenced TS/A cells 35 days after injection (n=12, P<0.01, Student's t-test). (D) Cell morphology after Ca²⁺ deprivation. Left panels: CTR 4T1 cells at time 0 and after 18 min, right panels: Fmr1 shRNA. Scale bar=50 μm. (E) E-cadherin-dependent adhesion assay. CTR and Fmr1 shRNA cells were plated on E-cadherin coated wells, let to adhere and stained with crystal violet (n=3, P<0.01, Student's t-test). (F) Extravasation test for Fmr1 silenced and control TS/A cells. The total number of lung metastasis 20 days after injection into the vein tail of WT syngenic mice is shown in the histogram (n=9, p<0.05, Student's t-test). Right panels show representative pictures of lung metastasis generated after (IV) injection of CTR and Fmr1 shRNA TS/A cells.

FIG. 7. FMRP expression and Fmr1 silencing in breast cancer cell lines.

(A) FMRP expression in mouse breast tumor cell lines (4T1, TS/A). FMRP levels were analysed by Western blot using specific FMRP antibodies (Ferrari et al., 2007) and normalised for GAPDH. Quantification is reported in the histogram as ratio FMRP/GAPDH where FMRP levels in 4T1 cells were considered 100%. (B) Fmr1 silencing in 4T1 cells. 4T1 cells were transiently transfected with five different shRNAs as well as with a scrambled shRNA (see Methods). FMRP levels were detected by Western blot, normalised to Vinculin and values reported in the histogram as ratio FMRP/Vinculin where FMRP levels in CTR cells were considered 100%. (C) 4T1 cells were stably transfected with different combination of three shRNAs against Fmr1 gene. FMRP levels were detected by Western blot and normalised to Vinculin or total proteins (Coomassie staining, data not shown). FMRP levels are expressed as a ratio to control cells (100%). (D) Primary tumor growth after orthotopic implantation of control (CTR) and Fmr1 silenced 4T1 cells. CTR and Fmr1 mRNA silenced cells (with combination of lentivirus 3/4) were orthotopically injected in WT syngenic mice (n=13). The graph represents tumor volume as a function of time after the injection. (E) Same as described in panel (C) using TS/A cell line. (F) Same as described in (D) using Fmr1 shRNA or CTR TS/A cells (n=12).

FIG. 8. FMRP does not affect cell growth in vitro.

(a-d) Growth rate of 4T1 and TS/A cells. Cells were grown in different media conditions (1% and 10% serum) and counted over a period of six days.

FIG. 9. FMRP expression in normal and tumor breast mouse tissues and effect of FMR1 silencing on tumors.

(A) Lane1, protein extracts from breast tissue of a 2 months old mouse; lanes 2 and 3, an example of protein extracts from two different murine breast tumors; lane 4, protein extracts from a 2 months old mouse brain. FMRP levels were analyzed by Western blot using specific FMRP antibodies (Ferrari et al., 2007). (B) FMRP signal was normalized for the ribosomal protein S6 and quantification reported in the histogram. (c) Proliferation rate in breast primary tumors induced by Fmr1 silenced or control TS/A cells. Histogram shows the percentage of cells positively stained for PCNA. p<0.05, Student's t-test. (d) Necrosis in breast primary tumors induced by Fmr1 silenced or control TS/A cells. Histogram shows the percentage of necrotica area, out of the total tumor area. p=0.0024, Student's t-test.

FIG. 10. In tumors E-cadherin expression is inversely correlated to FMRP levels.

(A) IHC (not shown) for FMRP and E-cadherin on tumors generated by control and Fmr1-silenced 4T1 cells. Histograms show the quantification (n=13, P<0.001, Student's test). Scale bars 200 μm. (B) IHC (not shown) for FMRP and Ecadherin on human non-metastatic (BC) and metastatic (metastatic BC) breast cancer. Histograms represent the percentage of FMRP and E-cadherin positive cells, respectively (n=9, P<0.05, Student's t-test). (C) E-cadherin cell surface levels in 4T1 shRNA and CTR shRNA cells (P<0.01, Student's t-test).

FIG. 11. E-cadherin expression in primary tumors.

Magnification of a representative IHC for E-cadherin on tumors generated by control (left panel) and Fmr1 shRNA 4T1 cells (right panel). Scale bars 20 μm.

FIG. 12. E-cadherin expression in lymphoblastoid cells from FXS patients.

E-cadherin expression in normal (AG), FXS (GM) and permutated (TF) lymphoblastoid cell lines dected by western blot. Upper left panel, Western Blot analysis of FMRP, E-cadherin and vimentin protein levels normalised to the ribosomal protein S6 (rpS6). Lower left panel, protein levels were plotted according to the increasing number of CGG repeats. Correlation coefficient R²=0,95. Right panels, quantification of FMRP, E-cadherin and vimentin levels after normalisation to rpS6. Shown is the ratio compared to cells from normal individuals (100%).

FIG. 13. FMRP regulates translation of E-cadherin mRNA in tumor cells.

(A) mRNAs associated to FMRP in CTR 4T1 cells. Upper panel, FMRP immunoprecipitation efficiency was shown by Western blot. Below, quantification of co-precipitating E-cadherin and Histone H3.3 and αTubulin mRNAs by RT-qPCR. Shown are the levels relative to the mock (IgG) immunoprecipitation. (B) mRNA translational efficiency analysis of 4T1 cells silenced with a control or an anti-FMRP shRNA. Upper panel, polysome-mRNPs distribution on a sucrose gradient (Napoli et al., 2008). mRNAs were extracted from the polysome (P, fractions 1-5) and mRNP (NP, fractions 6-10) regions of the sucrose gradient, and analysed by RT-qPCR. The lower panels show the [P]/[mRNP] ratio as a measure of translational activity.

FIG. 14. mRNAs associated to FMRP in 4T1 CTR cells and tumors.

(A) E-cadherin, Histone H3.3, αTubulin, Ferritin, Cytochrome C, and also vimentin, Msn, Igfbp4 and Dsp mRNAs were detected by RTPCR after FMRP immunoprecipitation from 4T1 CTR cells. Lane 1, marker (100 by DNA ladder); lane 2, input (1/50); lane 3, FMRP IP; lane 4, IgG IP; lane 5, PCR without cDNA. (B) E-cadherin and Histone H3.3 mRNAs were detected by RT-PCR after FMRP immunoprecipitation from tumor tissues generated by 4T1 CTR cell injection. Lanes 1-4 are as indicated in (A).

FIG. 15. Regulation of mRNA levels upon Fmr1 knockdown.

The amount of E-cadherin and Vimentin mRNA in total RNA extracts from 4T1 CTR and Fmr1 shRNA cell lines was measured by RT-qPCR. mRNA levels of Fmr1-silenced cells (using shRNA) versus control cells are shown.

FIG. 16. Tumor Kinetics.

Detection of GFP RNA levels in mice injected orthotopically with 4T1 CTR or Fmr1 shRNA cell lines carrying the GFP gene. The histogram shows that 5 weeks after injection less tumor cells are detected in the blood of mice injected with Fmr1 silenced cells.

### DETAILED DESCRIPTION

Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, N.Y. (1989); and Ausubel et al., Current Protocols in Molecular Biology (Supplement 47), John Wiley & Sons, New York (1999); “Ed Harlow and David Lane, Antibodies: A Laboratory Manual, Cold Spring Harbor Press, Plainsview, N.Y. (1988)”, for definitions and terms of the art. The definitions provided herein should not be construed to have a scope less than understood by a person of ordinary skill in the art.

The term “FMR1” as used herein refers to the fragile X mental retardation 1 gene (Gene ID: 2332 for the human gene), also known as POF, FMRP, POF1, FRAXA, and MGC87458, and its products. The “FMR1 gene product” as used herein typically refers to what is transcribed or translated from the FMR1 gene, such as Fmr1 mRNA and FMRP protein. The different isoforms or variants of Fmr1 mRNA and the resulting FMRP isoforms or variants (e.g. Verkerk et al., 1993; Ashley et al., 1993; Verheij et al., 1995) are envisaged within the term FMR1 gene product. Fragments of a FMR1 gene product are also envisaged, as long as they are functionally active. Indeed, typically the FMR1 gene (or gene product) to be detected or inhibited will be a functionally active gene, e.g. not a Fmr1 mRNA that is unstable due to increased triplet repeats, as found in Fragile X syndrome.

A “FMR1 inhibitor” as used herein refers to a substance that can interfere with the function of the FMR1 gene product, either at the DNA level (by inhibiting the formation of FMR1 gene product, i.e. by preventing or interfering with transcription), at the RNA level (by neutralizing or destabilizing mRNA to prevent or interfere with translation) or at the protein level (by neutralizing or inhibiting FMRP protein).

According to a first aspect, provided herein are methods of assessing metastatic potential of a tumour in a subject, comprising determining the levels of FMR1 gene product in said tumour. “Metastatic potential” as used herein refers to the probability that the tumour will metastasize in the future. Typically, the tumour will not have metastasized yet at the time of determining metastatic potential.

Particularly, the tumour of which the metastatic potential is to be determined, is a solid tumour. The levels of FMR1 gene product may be determined from the whole tumour, or from any subsection from within the solid tumour. Most particularly, the FMR1 gene product levels will be determined from the invasive front of the tumour, at the tumour-host interface. Of note, determining the levels of FMR1 gene product will typically not be done on the subject self, but in vitro. For instance, a biopsy or other sample taken from the tumor may be provided, and the analysis of the FMR1 gene product levels can happen on the tumour sample. Again, the biopsy or other sample may be taken from any subsection, particularly from the invasive front of the tumour.

Typically, the methods of assessing metastatic potential provided herein will further include a step involving correlating the levels of FMR1 gene product to the risk of metastasis, particularly correlating increased levels of FMR1 gene product to increased risk of metastasis. However, the reverse can also be true: concluding from an observation that the FMR1 gene product levels are not increased, or are decreased, or are even absent, in the tumour, that there is no increased risk of metastasis, or in some instances even a decreased risk of metastasis.

Increased levels of FMRP gene product are typically increased versus a control. The skilled person is capable of picking the most relevant control. This will typically also depend on the nature of the tumour studied, the sample(s) that is/are available, and so on. Suitable controls include, but are not limited to, a cancer-free sample of the tissue where the solid tumour is located (e.g. a breast tissue sample in case of breast cancer, optionally, but not necessarily, from the same subject), a tumour sample from a tumour that is known not to metastasize (particularly, but not necessarily, from a tumour located in the same or similar type of tissue, e.g. a non-metastasizing breast tumour in case of breast cancer), or a set of clinical data on average FMR1 gene product levels in non-metastasizing tumours. As is evident from the foregoing, the control may be from the same subject, or from one or more different subjects or derived from clinical data. Optionally, the control is matched for e.g. sex, age etc.

With ‘increased’ levels of FMR1 gene product as mentioned herein, it is meant levels that are higher than are normally present. Typically, this can be assessed by comparing to control. According to particular embodiments, increased levels of FMR1 are levels that are 10%, 20%, 25%, 30%, 40%, 50%, 60%, 70%, 75%, 80%, 90%, 100%, 150%, 200% or even more high than those of the control. According to further particular embodiments, it means that FMR1 gene product is present, whereas it normally (or in control) is not expressed, or is expressed at very low or barely detectable levels. In other words, in these embodiments detecting the presence of FMR1 gene product is equivalent to detecting increased levels of FMR1 gene product. According to yet further particular embodiments, it means that FMR1 gene product is present, whereas in the majority of tumour-free samples, taken as a control, it is not. The skilled person will appreciate that the exact levels by which FMR1 gene product needs to be higher in order to allow a reliable and reproducible diagnosis may depend on the type of tumour tested and of which product (mRNA, protein) the levels are assessed. However, assessing the correlation itself is fairly straightforward, particularly as FMRP levels in most tissues are low or even absent—see example 1.

Instead of looking at increased levels compared to a negative control, the skilled person will appreciate that the reverse, comparing to a positive control, can also be done. Thus, if the FMR1 gene product levels measured in the tumour sample are similar to those of a ‘control’ tumour known to metastasize, (or are e.g. comparable to FMR1 gene product levels found in a clinical data set of aggressive tumours), this may be considered equivalent to increased FMR1 gene product levels compared to a negative control, and be correlated to an increased risk of metastasis. In the other case, if FMR1 gene product levels are lower than those of a positive control, this can be said not to correlate with an increased risk of metastasis, or even to be correlated with a decreased risk of metastasis. For decreased levels of FMR1 gene product levels compared to a positive control, the considerations about increased levels of FMR1 gene product apply mutatis mutandis. Of course, FMR1 gene product levels may be compared to both a negative and a positive control in order to increase accuracy of the diagnosis.

The FMR1 gene product whose levels are determined will typically be Fmr1 mRNA and/or FMRP protein. When Fmr1 mRNA is chosen as the (or one of the) FMR1 gene product whose levels are determined, this can be the total of all Fmr1 mRNA isoforms, or one or more specific mRNAs. Multiple alternatively spliced transcript variants that encode different protein isoforms and which are located in different cellular locations have been described for the FMR1 gene (Verkerk et al., 1993; Ashley et al., 1993). However, only a minority of these and their corresponding protein products are actually detected in various tissues (Verheij et al., 1995; Oostra and Chiurazzi, 2001). Thus, depending on e.g. the tissue of origin, a skilled person will readily be able to determine of which isoform(s) he will determine the levels if total FMR1 mRNA is not determined. Examples include, but are not limited to, ISO1 (the longest transcript encoding the longest protein), ISO6 (lacking an alternate segment and using a different splice site in the 3′ coding region which shifts the reading frame, compared to variant ISO1), ISO7 (lacking an alternate segment, compared to variant ISO1), ISO9 (lacking an alternate segment and using a different splice site in the 3′ coding region, compared to variant ISO1), and ISO12 (lacking two alternate segments and using a different splice site which changes the reading frame, compared to variant ISO1).

Alternatively or additionally, the FMR1 gene product of which the levels are determined may be FMRP protein. As protein is translated from mRNA and the mRNA exists in multiple isoforms, the same considerations apply: the total FMRP levels may be determined, or those of specific isoforms only (e.g. using an antibody against the different C-termini). Most particularly, all FMRP protein isoforms may be detected (e.g. using an antibody against a common epitope). Of note, it is envisaged as well that both FMR1 mRNA and FMRP protein are determined. In this case, the isoforms to be detected can be all isoforms for both mRNA and protein, identical isoforms (wholly overlapping), or different isoforms (partly or not overlapping), depending on the setup of the experiment. With identical isoforms, it is meant that the mRNA isoform encodes for the corresponding protein isoform. However, it is known that the number of FMRP protein isoforms detected in tissue is generally lower than the number of possible mRNA transcripts (and thus of protein isoforms) (Oostra and Chiurazzi, 2001).

Also envisaged is the detection of FMRP protein with specific post-translational modifications, either within the whole FMRP pool or the selective detection of such modified proteins. Examples of such modified FMRP proteins include, but are not limited to, methylated FMRP, phosphorylated FMRP, ubiquitinylated FMRP, glycosylated FMRP or any combination thereof.

Suitable FMRP antibodies for detection include e.g. those described by Ferrari et al. (Ferrari et al., 2007) or those of Brown et al. (Brown et al., 2001) or a commercially available Ab against the N-terminus of FMRP (1C3, available from Chemicon).

In principle, the methods can be applied for any type of tumour, particularly any solid tumour, for which the risk of metastasis is to be determined. Most particularly, however, the tumour is selected from the group of breast cancer, colon cancer, and bladder cancer. Particularly envisaged is to apply the methods provided herein for assessing the metastatic potential of breast cancer. Most particularly, the methods can be applied for assessing the metastatic potential of lymph node negative breast cancer. The lymph node status can be assessed separately (i.e. in a different assay, or at a different time point) from the determination of FMR1 gene product levels, or can be done simultaneously or concomitantly with the determination of FMR1 gene product levels.

According to a further aspect, the positive correlation between levels of functional FMR1 gene product and metastatic potential can be exploited beneficially. That is to say, not only is it possible to assess metastatic potential by determining the levels of FMR1 gene product, but it is also feasible to reduce this metastatic potential by lowering functional FMRP levels. Accordingly, methods of preventing and/or treating metastasis of a tumour in a subject are provided, comprising inhibiting functional expression of the FMR1 gene in said subject, e.g. by administering an FMR1 inhibitor to the subject.

With “functional expression” of the FMR1 gene, it is meant the transcription and/or translation of functional FMR1 gene product—as opposed to e.g. the defunct transcripts and lack of functional gene product observed in Fragile X syndrome. “Inhibition of functional expression” can be achieved at three levels. First, at the DNA level, e.g. by removing or disrupting the FMR1 gene, or preventing transcription to take place (in both instances preventing synthesis of the FMR1 gene product). Second, at the RNA level, e.g. by preventing efficient translation to take place—this can be through destabilization of the mRNA so that it is degraded before translation occurs from the transcript, or by hybridizing to the mRNA. Third, at the protein level, e.g. by binding to the protein, inhibiting its function, and/or marking the protein for degradation.

If inhibition is to be achieved at the DNA level, this may be done using gene therapy to knock-out or disrupt the FMR1 gene. As used herein, a “knock-out” can be a gene knockdown or the gene can be knocked out by a mutation such as, a point mutation, an insertion, a deletion, a frameshift, or a missense mutation by techniques known in the art, including, but not limited to, retroviral gene transfer. Another way in which genes can be knocked out is by the use of zinc finger nucleases. Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms.

Particularly, the knock-out of the FMR1 gene is limited to the tissue where the solid tumour is located, most particularly, the knock-out is limited to the tumour itself, and FMR1 is not inhibited in the host subject.

Apart from tissue-specific inhibition of FMR1 gene product function, the inhibition may also be temporary (or temporally regulated).

Temporally and tissue-specific gene inactivation may for instance also be achieved through the creation of transgenic organisms expressing antisense RNA, or by administering antisense RNA to the subject. An antisense construct can be delivered, for example, as an expression plasmid, which, when transcribed in the cell, produces RNA that is complementary to at least a unique portion of the cellular Fmr1 mRNA.

A more rapid method for the inhibition of gene expression is based on the use of shorter antisense oligomers consisting of DNA, or other synthetic structural types such as phosphorothiates, 2′-O-alkylribonucleotide chimeras, locked nucleic acid (LNA), peptide nucleic acid (PNA), or morpholinos. With the exception of RNA oligomers, PNAs and morpholinos, all other antisense oligomers act in eukaryotic cells through the mechanism of RNase H-mediated target cleavage. PNAs and morpholinos bind complementary DNA and RNA targets with high affinity and specificity, and thus act through a simple steric blockade of the RNA translational machinery, and appear to be completely resistant to nuclease attack. An “antisense oligomer” refers to an antisense molecule or anti-gene agent that comprises an oligomer of at least about 10 nucleotides in length. In embodiments an antisense oligomer comprises at least 15, 18 20, 25, 30, 35, 40, or 50 nucleotides. Antisense approaches involve the design of oligonucleotides (either DNA or RNA, or derivatives thereof) that are complementary to an mRNA encoded by polynucleotide sequences of FMR1. Antisense RNA may be introduced into a cell to inhibit translation of a complementary mRNA by base pairing to it and physically obstructing the translation machinery. This effect is therefore stoichiometric. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense polynucleotide sequences, a single strand of the duplex DNA may thus be tested, or triplex formation may be assayed. The ability to hybridize will depend on both the degree of complementarity and the length of the antisense polynucleotide sequence. Generally, the longer the hybridizing polynucleotide sequence, the more base mismatches with an RNA it may contain and still form a stable duplex (or triplex, as the case may be). One skilled in the art can ascertain a tolerable degree of mismatch by use of standard procedures to determine the melting point of the hybridized complex. Oligomers that are complementary to the 5′ end of the message, e.g., the 5′ untranslated region (UTR) up to and including the AUG translation initiation codon, should work most efficiently at inhibiting translation. However, sequences complementary to the 3′ UTR of mRNAs have recently been shown to be effective at inhibiting translation of mRNAs as well (Wagner, R. (1994) Nature 372, 333-335). Therefore, oligomers complementary to either the 5′, 3′ UTRs, or non-coding regions of a FMR1 gene could be used in an antisense approach to inhibit translation of said endogenous mRNA encoded by FMR1 polynucleotides. Oligomers complementary to the 5′ UTR of said mRNA should include the complement of the AUG start codon. Antisense oligomers complementary to mRNA coding regions are less efficient inhibitors of translation but could be used in accordance with the invention. Whether designed to hybridize to the 5′, 3′ or non-coding region of a said mRNA, antisense oligomers should be at least 10 nucleotides in length, and are preferably oligomers ranging from 15 to about 50 nucleotides in length. In certain embodiments, the oligomer is at least 15 nucleotides, at least 18 nucleotides, at least 20 nucleotides, at least 25 nucleotides, at least 30 nucleotides, at least 35 nucleotides, at least 40 nucleotides, or at least 50 nucleotides in length. A related method uses ribozymes instead of antisense RNA. Ribozymes are catalytic RNA molecules with enzyme-like cleavage properties that can be designed to target specific RNA sequences. Successful target gene inactivation, including temporally and tissue-specific gene inactivation, using ribozymes has been reported in mouse, zebrafish and fruitflies. RNA interference (RNAi) is a form of post-transcriptional gene silencing. The phenomenon of RNA interference was first observed and described in Caenorhabditis elegans where exogenous double-stranded RNA (dsRNA) was shown to specifically and potently disrupt the activity of genes containing homologous sequences through a mechanism that induces rapid degradation of the target RNA. Several reports describe the same catalytic phenomenon in other organisms, including experiments demonstrating spatial and/or temporal control of gene inactivation, including plant (Arabidopsis thaliana), protozoan (Trypanosoma bruceii), invertebrate (Drosophila melanogaster), and vertebrate species (Danio rerio and Xenopus laevis). The mediators of sequence-specific messenger RNA degradation are small interfering RNAs (siRNAs) generated by ribonuclease III cleavage from longer dsRNAs. Generally, the length of siRNAs is between 20-25 nucleotides (Elbashir et al. (2001) Nature 411, 494-498). The siRNA typically comprise a sense RNA strand and a complementary antisense RNA strand annealed together by standard Watson Crick base pairing interactions (hereinafter “base paired”). The sense strand comprises a nucleic acid sequence that is identical to a target sequence contained within the target mRNA. The sense and antisense strands of the present siRNA can comprise two complementary, single stranded RNA molecules or can comprise a single molecule in which two complementary portions are base paired and are covalently linked by a single stranded “hairpin” area (often referred to as shRNA). The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non native environment such as, for example, a cell into which the siRNA has been delivered.

The siRNAs of the invention can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non nucleotide material, such as to the end(s) of the siRNA or to one or more internal nucleotides of the siRNA, including modifications that make the siRNA resistant to nuclease digestion.

One or both strands of the siRNA of the invention can also comprise a 3′ overhang. A “3′ overhang” refers to at least one unpaired nucleotide extending from the 3′ end of an RNA strand. Thus, in one embodiment, the siRNA of the invention comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about four nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides.

Alternatively, substitution of pyrimidine nucleotides by modified analogues, e.g., substitution of uridine nucleotides in the 3′ overhangs with 2′ deoxythymidine, is tolerated and does not affect the efficiency of RNAi degradation. In particular, the absence of a 2′ hydroxyl in the 2′ deoxythymidine significantly enhances the nuclease resistance of the 3′ overhang in tissue culture medium.

The siRNAs of the invention can be targeted to any stretch of approximately 19 to 25 contiguous nucleotides in any of the target Fmr1 mRNA sequences (the “target sequence”), of which examples are given in the application. Techniques for selecting target sequences for siRNA are well known in the art. Thus, the sense strand of the present siRNA comprises a nucleotide sequence identical to any contiguous stretch of about 19 to about 25 nucleotides in the target mRNA.

The siRNAs of the invention can be obtained using a number of techniques known to those of skill in the art. For example, the siRNAs can be chemically synthesized or recombinantly produced using methods known in the art. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the invention from a plasmid include, for example, the U6 or H1 RNA pol Ill promoter sequences and the cytomegalovirus promoter. Selection of other suitable promoters is within the skill in the art. The recombinant plasmids of the invention can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly, e.g. in breast tissue or in neurons.

The siRNAs of the invention can also be expressed intracellularly from recombinant viral vectors. The recombinant viral vectors comprise sequences encoding the siRNAs of the invention and any suitable promoter for expressing the siRNA sequences. Suitable promoters include, for example, the U6 or H1 RNA pol Ill promoter sequences and the cytomegalovirus promoter. Selection of other suitable promoters is within the skill in the art. The recombinant viral vectors of the invention can also comprise inducible or regulatable promoters for expression of the siRNA in the tissue where the tumour is localized.

As used herein, an “effective amount” of the siRNA is an amount sufficient to cause RNAi mediated degradation of the target mRNA, or an amount sufficient to inhibit the progression of metastasis in a subject. RNAi mediated degradation of the target mRNA can be detected by measuring levels of the target mRNA or protein in the cells of a subject, using standard techniques for isolating and quantifying mRNA or protein as described above.

One skilled in the art can readily determine an effective amount of the siRNA of the invention to be administered to a given subject, by taking into account factors such as the size and weight of the subject; the extent of the disease penetration; the age, health and sex of the subject; the route of administration; and whether the administration is regional or systemic. Generally, an effective amount of the siRNA of the invention comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of siRNA can be administered.

Recently it has been shown that morpholino antisense oligonucleotides in zebrafish and frogs overcome the limitations of RNase H-competent antisense oligonucleotides, which include numerous non-specific effects due to the non target-specific cleavage of other mRNA molecules caused by the low stringency requirements of RNase H. Morpholino oligomers therefore represent an important new class of antisense molecule. Oligomers of the invention may be synthesized by standard methods known in the art. As examples, phosphorothioate oligomers may be synthesized by the method of Stein et al. (1988) Nucleic Acids Res. 16, 3209-3021), methylphosphonate oligomers can be prepared by use of controlled pore glass polymer supports (Sarin et al. (1988) Proc. Natl. Acad. Sci. USA. 85, 7448-7451). Morpholino oligomers may be synthesized by the method of Summerton and Weller U.S. Pat. Nos. 5,217,866 and 5,185,444.

An example of a suitable FMR1 shRNA are for instance the two recently used in a paper by Silva et al. (Silva et al., 2009)

The FMR1 gene product inhibitor may also be an inhibitor of FMRP protein. A typical example thereof is an anti-FMRP antibody.

The term ‘antibody’ or ‘antibodies’ relates to an antibody characterized as being specifically directed against FMRP or any functional derivative thereof, with said antibodies being preferably monoclonal antibodies; or an antigen-binding fragment thereof, of the F(ab′)₂, F(ab) or single chain Fv type, or any type of recombinant antibody derived thereof. These antibodies of the invention, including specific polyclonal antisera prepared against FMRP or any functional derivative thereof, have no cross-reactivity to other proteins. The monoclonal antibodies of the invention can for instance be produced by any hybridoma liable to be formed according to classical methods from splenic cells of an animal, particularly of a mouse or rat immunized against FMRP or any functional derivative thereof, and of cells of a myeloma cell line, and to be selected by the ability of the hybridoma to produce the monoclonal antibodies recognizing FMRP or any functional derivative thereof which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies according to this embodiment of the invention may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also fragments derived from these monoclonal antibodies such as Fab, F(ab)′₂ and scFv (“single chain variable fragment”), providing they have retained the original binding properties, form part of the present invention. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies involved in the invention can be labeled by an appropriate label of the enzymatic, fluorescent, or radioactive type. In a particular embodiment said antibodies against FMRP or a functional fragment thereof are derived from camels. Camel antibodies are fully described in WO94/25591, WO94/04678 and in WO97/49805. Processes are described in the art which make it possible that antibodies can be used to hit intracellular targets. Since FMRP is an intracellular target, the antibodies or fragments thereof with a specificity for FMRP must be delivered into the cells. One such technology uses lipidation of the antibodies. The latter method is fully described in WO94/01131 and these methods are herein incorporated by reference. Another method is by fusing the antibody to cell-penetrating peptides (Chen and Harrison, Biochem Soc Trans. 2007).

If the tumour is located in the brain, the inhibitor should be able to pass the blood-brain barrier. Technologies of modifying antibodies to pass the blood-brain barrier are well known to the skilled person.

Other inhibitors of FMRP include, but are not limited to, peptide inhibitors of FMRP, peptide-aptamer (Tomai et al., J Biol Chem. 2006) inhibitors of FMRP, and protein interferors as described in WO2007/071789, incorporated herein by reference.

Small molecule inhibitors, e.g. small organic molecules, and other drug candidates can be obtained, for example, from combinatorial and natural product libraries.

In summary, an “inhibitor of FMR1” as used herein can be, but is not limited to: a chemical, a small molecule, a drug, an antibody, a peptide, a secreted protein, a nucleic acid (such as DNA, RNA, a polynucleotide, an oligonucleotide or a cDNA) or an antisense RNA molecule, a ribozyme, an RNA interference nucleotide sequence, an antisense oligomer, a zinc finger nuclease or a morpholino.

Inhibition of FMR1 gene product does not necessarily mean complete ablation of FMR1 function, although this is envisaged as well. Particularly with antisense RNA and siRNA, but with antibodies as well, it is known that inhibition is often partial inhibition rather than complete inhibition. However, lowering functional FMR1 gene product levels will have a beneficial effect even when complete inhibition is not achieved—particularly in those cases where the FMR1 gene is also expressed in the non-tumoral tissue, albeit to a lesser extent. Thus, according to particular embodiments, the inhibition will result in a decrease of 10%, 20%, 25%, 30%, 40%, 50%, 60%, 70%, 75%, 80%, 90% or up to 100% of functional FMR1 gene product. Methods of measuring the levels of functional FMR1 gene product are known to the skilled person, and he can measure these before and after the addition of the inhibitor to assess the decrease in levels of functional FMR1 gene product.

The tumours that can be treated by inhibiting FMR1 are the same as described for the methods of assessing metastatic potential. Thus, while any tumour in principle can be treated, most particularly, the tumour is selected from the group of breast cancer, colon cancer, bladder cancer and stomach cancer. Particularly envisaged is to apply the methods of treatment provided herein for treatment and/or prevention of metastasis of breast cancer. Although it can be used for treatment of all breast cancer, correlation with high FMRP levels is most notable for lymph node negative breast cancers, so FMR1 gene product inhibition as treatment is most particularly envisaged for this class of cancers.

According to a further aspect, an FMR1 inhibitor as described herein is provided for use as a medicament. Particularly envisaged is the use of a siRNA against Fmr1 mRNA for use as a medicament. According to further embodiments, an FMR1 inhibitor is provided for use in treatment and/or prevention of metastasis of a tumour. (Again, tumours and inhibitors are as described herein). Accordingly, also provided is a a pharmaceutical composition comprising an effective amount of at least one FMR1 inhibitor. Most particularly, said inhibitor is a siRNA against Fmr1, such as in a most particular embodiment an isolated siRNA comprising a sense RNA strand and an antisense RNA strand, wherein the sense and the antisense RNA strands form an RNA duplex, and wherein the sense RNA strand comprises a nucleotide sequence identical to a target sequence of about 19 to about 25 contiguous nucleotides in the Fmr1 mRNA.

It is to be understood that although particular embodiments, specific configurations as well as materials and/or molecules, have been discussed herein for cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

### FMRP Expression in Cancer

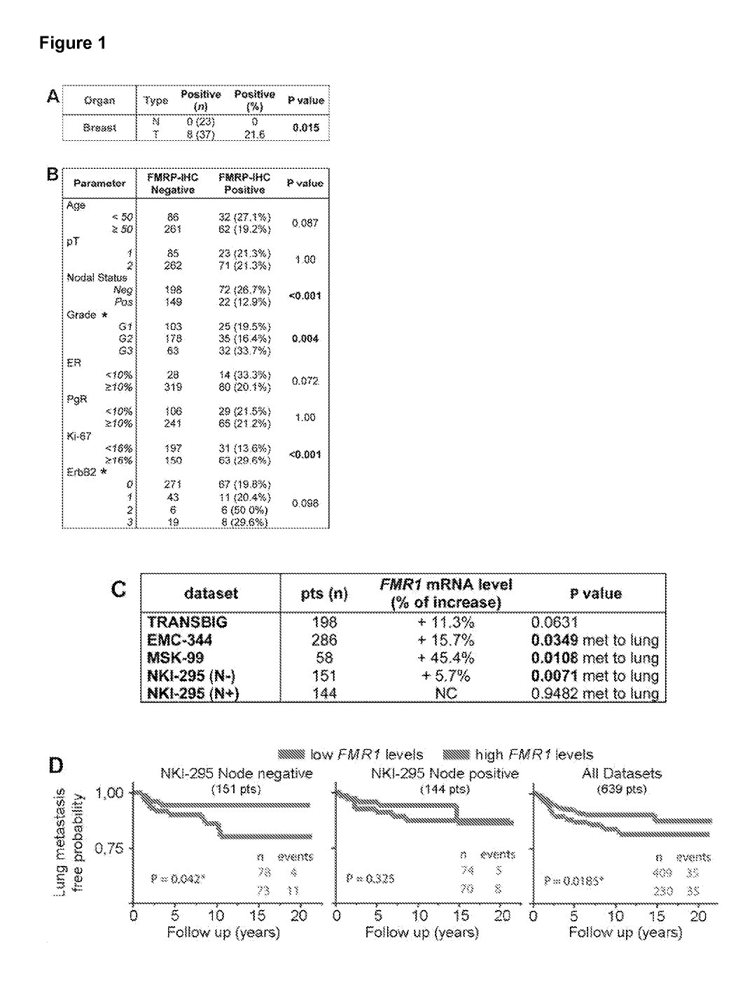

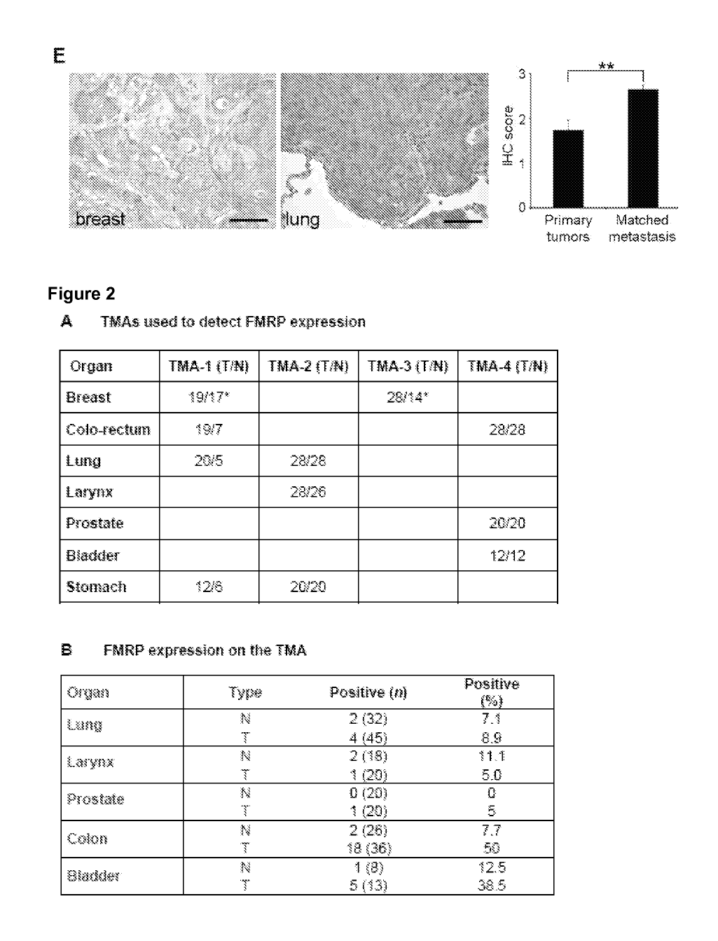

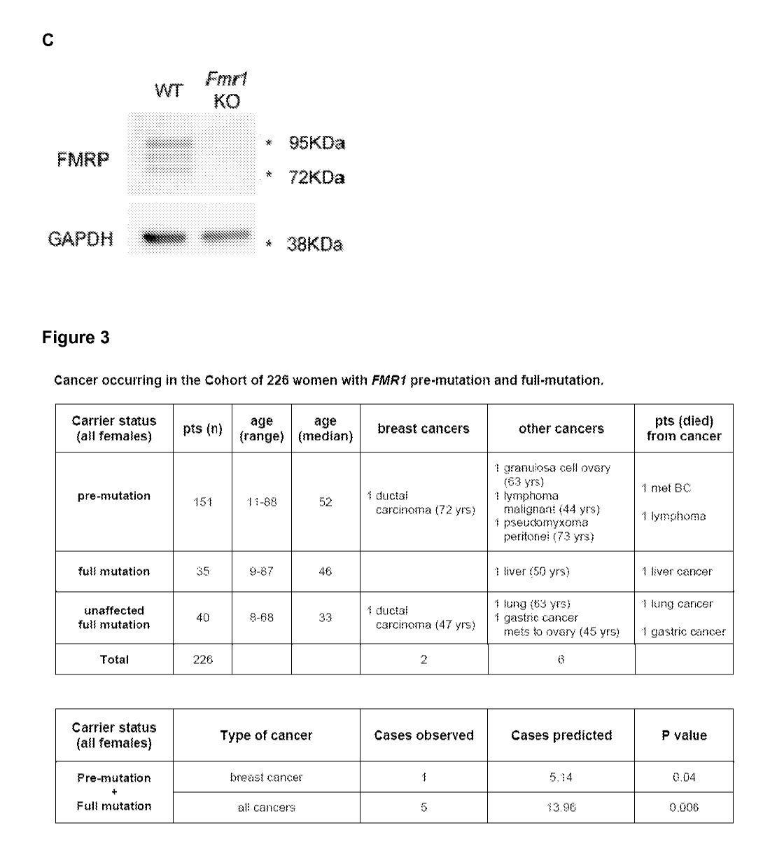

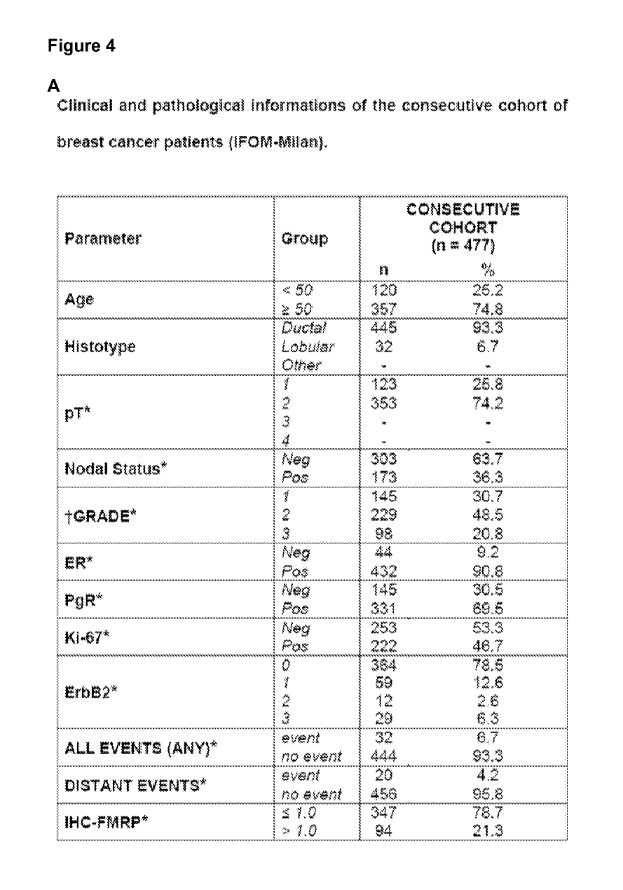

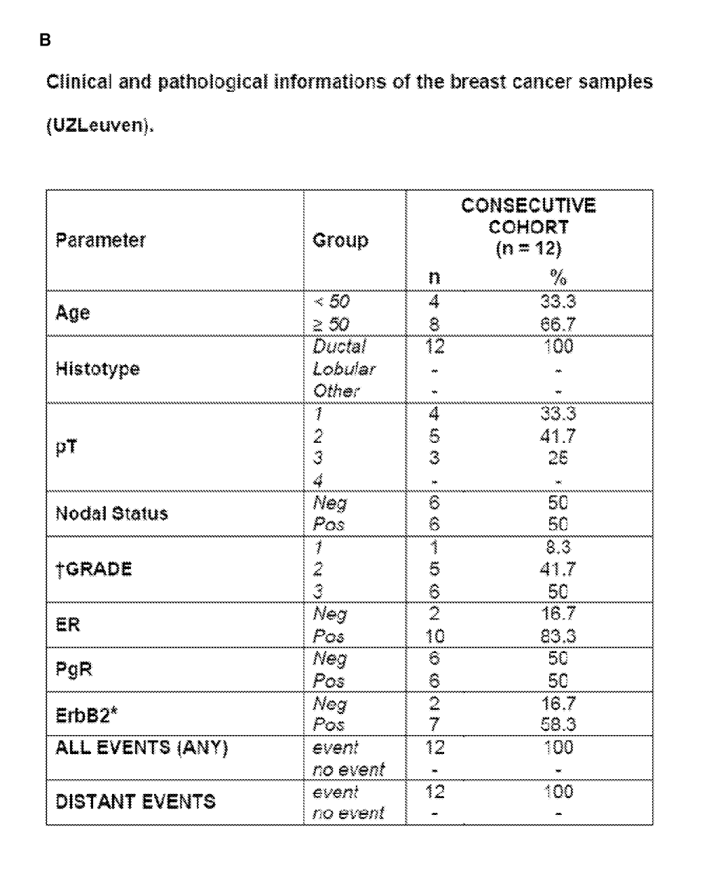

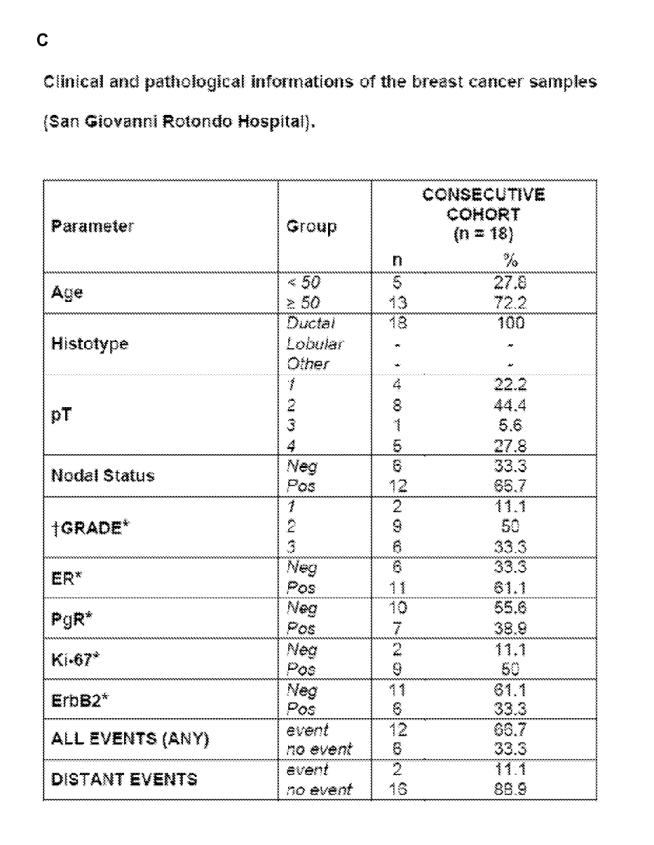

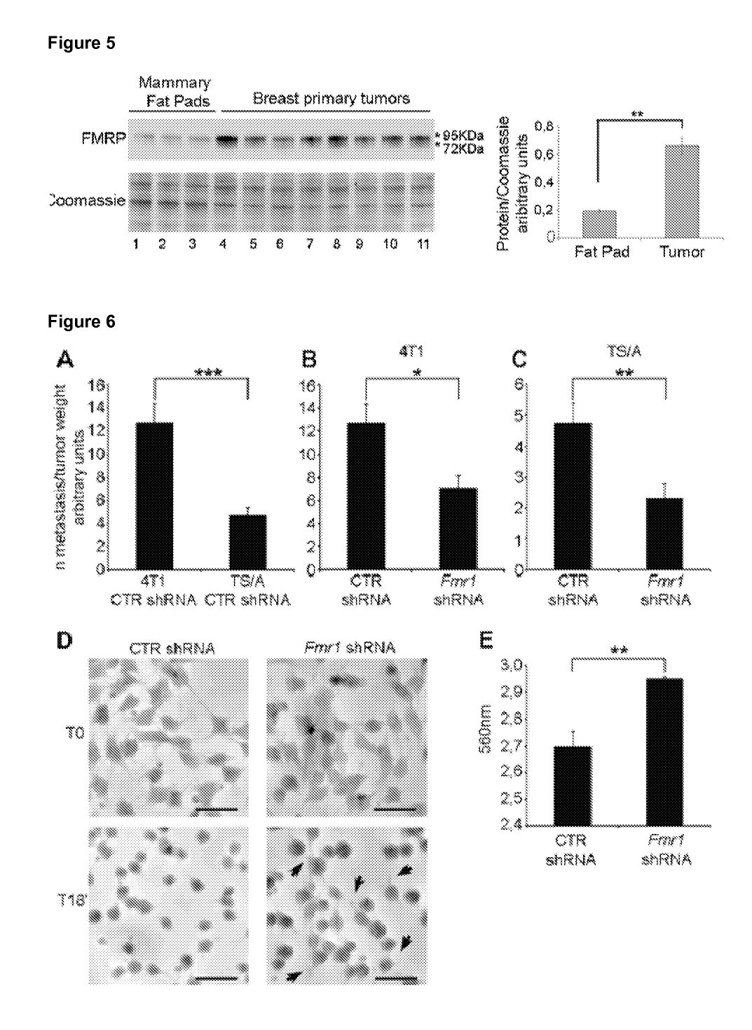

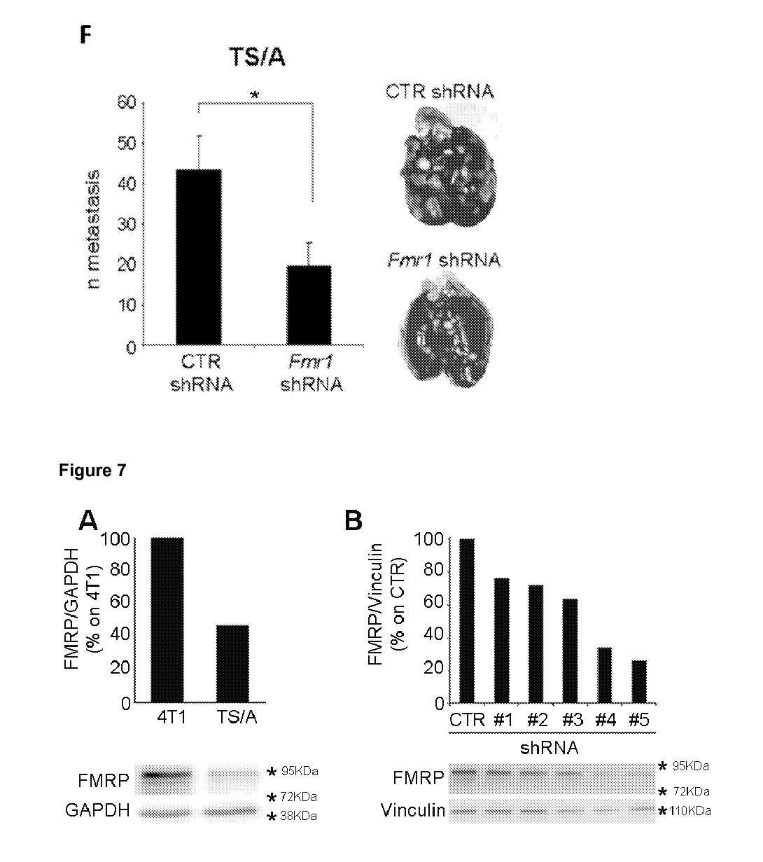

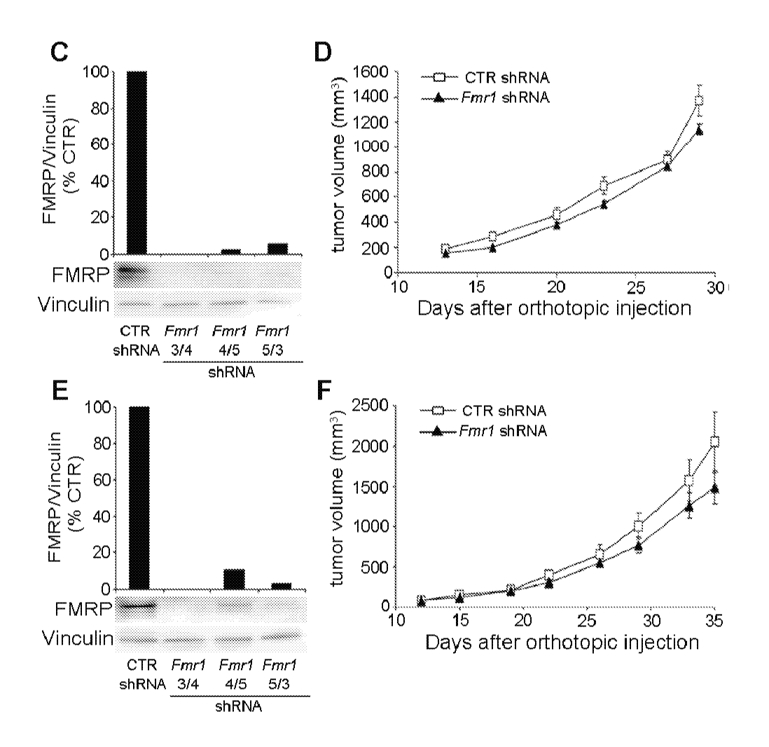

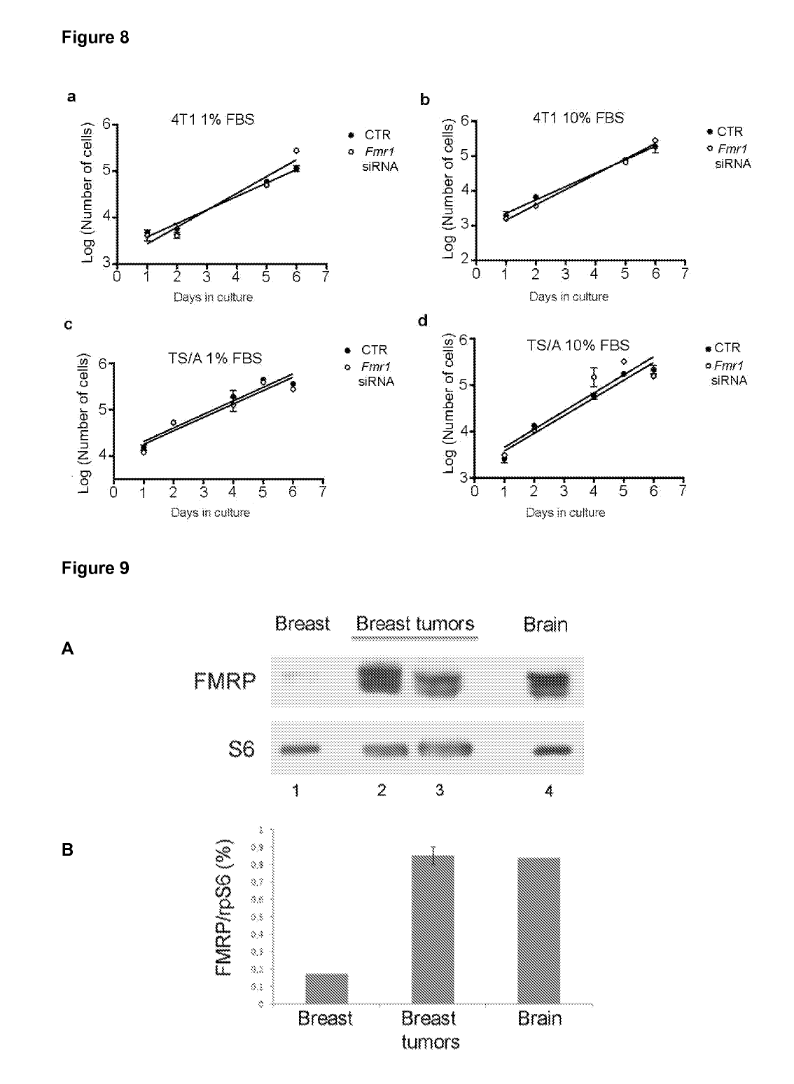

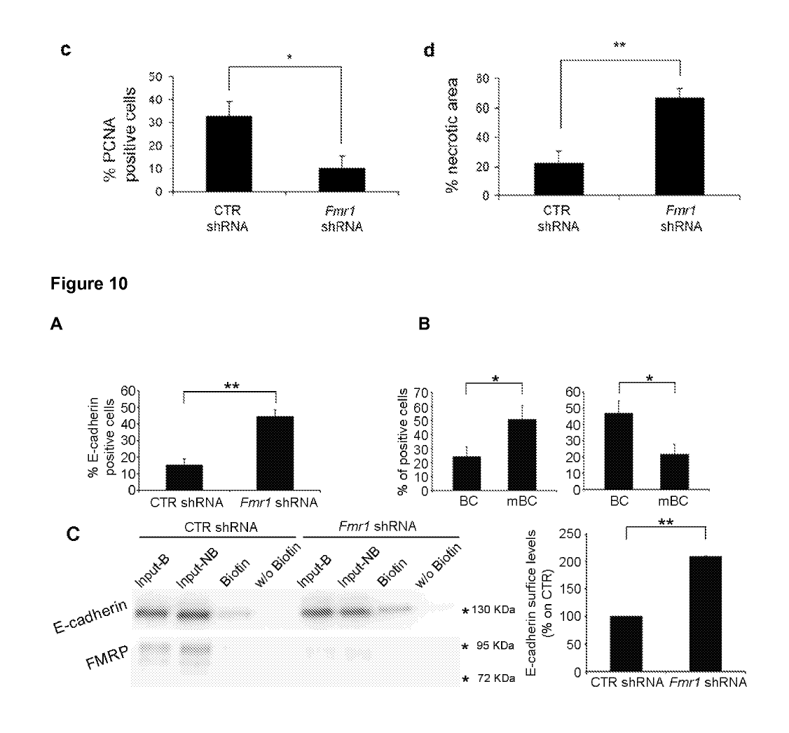

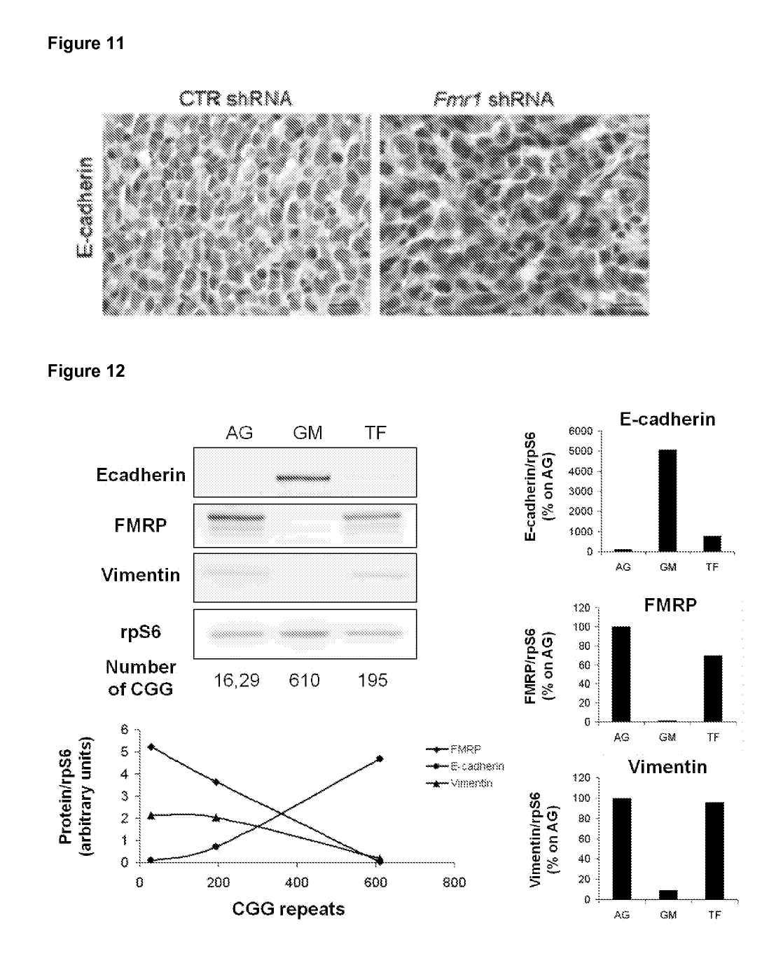

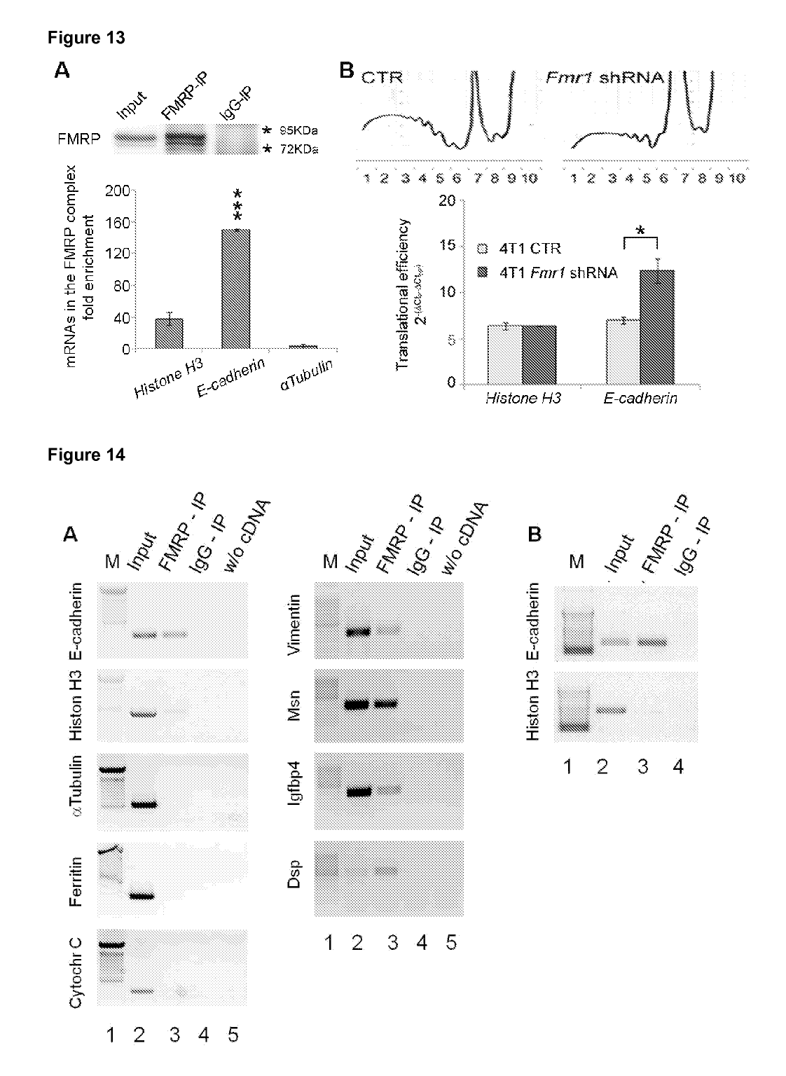

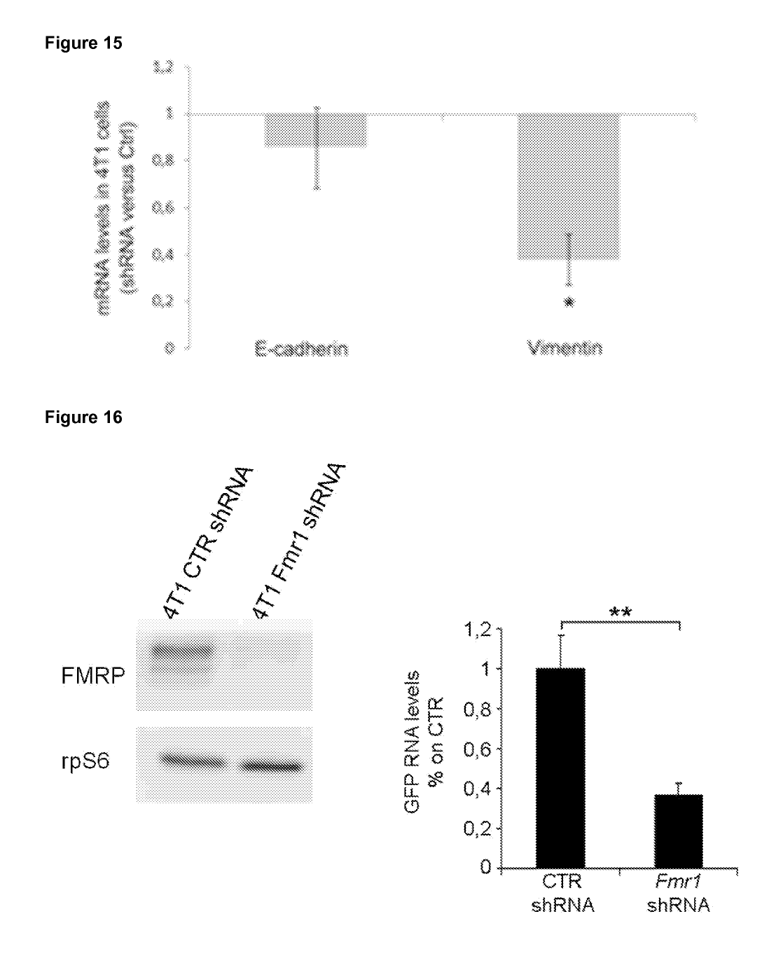

FMRP protein levels were examined in human cancer tissues previously characterized (Capra et al., 2006; Confalonieri et al., 2009) in the previously described human cancer tissue microarray (TMA, results shown in FIGS. 1A and 2B, description in FIG. 2A). FMRP expression on tissue microarrays (positive immunostaining with a cut-off >1, see Methods for details) was significantly changed in different tumor tissue types including breast, colon, and bladder. FMRP expression was significantly increased in breast tumor tissue as compared to normal tissues (FIG. 1A, P=0.015; antibody specificity is demonstrated in Ferrari et al., 2007 and FIG. 2C, as well as data not shown). Furthermore high levels of FMRP were detected in the invasive front of the tumor (not shown). In other cancer tissue types investigated in this study the difference between FMRP levels in normal and tumour tissue is typically less pronounced (FIG. 2B), although FMRP is increased in other tumours as well, most notably in colon cancer (P<0.01). These findings show that FMRP protein expression is altered in different human carcinomas and that its subversion might be relevant in multiple cancer types.

To further validate our findings in the context of FXS, the occurrence of cancer was studied in a cohort of 226 women with full mutation and pre-mutation FMR1 alleles. FMRP is completely lacking in full mutations and is decreased in pre-mutation carriers. Five patients with cancer were identified (excluding unaffected full mutations, n=186), significantly less than the expected 13.96 (P=0.006, FIG. 3). Only one case of breast cancer was present compared to an expected value of 5.14 (P=0.04). These findings support the hypothesis that reduction or absence of FMRP protects against cancer.

Using a previously characterised (Capra et al., 2006) large collection of breast cancer tissues, FMRP was found over-expressed in more than 20% of breast tumor samples (FIGS. 1B and 4A), in line with the percentage of positive breast tumors identified in the multi-tumor TMA survey (FIG. 1a). FMRP correlated with negative lymph node status (P<0.001), suggesting that FMRP overexpression may favor a systemic cancer development without lymph involvement. Remarkably, FMRP expression was significantly correlated with high tumor grade (G3, P=0.004) and high proliferation index (Ki67, P<0.001) (FIG. 1B), both of them prognostic indicators of metastatic disease (Elston and Ellis, 1991; Fitzgibbons et al., 2000; Goldhirsch et al., 2001). Further, FMRP tends to be more expressed in estrogen receptor (ER)-negative breast cancer (P=0.072, FIG. 1B), which is also more likely to metastasize. Importantly, histopathology analysis of paired cases (n=5) of primary tumors and metastases showed a tendency of increased FMRP expression in the metastatic tissue (not shown) suggesting a potential biological role for FMRP in tumor dissemination.

To evaluate whether FMRP expression indeed correlates with metastatic cancer, we performed a gene expression analysis on five available breast cancer datasets (FIG. 1C). The TRANSBIG (Desmedt et al., 2007) cohort of 198 breast cancer patients analysis revealed an increased expression of FMR1 mRNA, encoding for FMRP, in metastatic primary tumors to distal organs versus non-metastic primary tumors (P=0.0631). In two independent cohorts that contain only samples with negative nodal status, EMC-344 (Wang et al., 2005) and MSK-99 (Minn et al., 2005), FMR1 expression was significantly increased in primary tumors that metastasize to lung (FIG. 1C, n=344, P=0.0349 and 0.0108, respectively). Also in the NKI-295 (van de Vijver et al., 2002) dataset we found that FMR1 expression is correlated with lung metastases in the lymph node-negative subpopulation (FIG. 1C, n=151, P=0.0071 and FIG. 1D P=0.042). Conversely, in lymph node positive primary tumors, FMR1 mRNA expression neither correlates with tumors metastatic to lungs (FIG. 1C, P=0.9482) nor with the probability of developing lung metastasis (FIG. 1D, P=0.325). The three data sets EMC-344, MSK-99 and NKI-295 showed that high levels of FMR1 increase the probability of lung metastasis (FIG. 1D, n=639, P=0.0185). Metastasis to other distant organs did not reach significance (not shown), but this may be explained both by the proclivity that breast carcinomas have for metastasis to lung tissue (e.g. Fidler, 2003) and the enrichment of lung metastasizing tumours in the dataset (Minn et al., 2005) and thus is probably not an FMRP-related effect. Multivariate Cox proportional hazard analysis of the three cohorts revealed that increased FMR1 mRNA expression has a hazard ratio of 1.21 for lymph node-negative tumors that metastasize to the lungs (95% CI 1.02-1.45, P=0.0293). The same dataset analyzed for Estrogen Receptor mRNA revealed a hazard ratio of 1.51 (95% CI 1.27-1.85, P<0.0001, Likelihood Ratio Test). Furthermore, histopathology analysis of paired cases of breast primary tumors and distal metastases (FIGS. 1E, 4A and 4B) showed an increased FMRP expression in the metastatic tissues (n=17, P=0.0010), suggesting a potential role for FMRP in tumor cell dissemination to distal sites. These findings suggest a possible correlation between FMRP expression and spreading to distant sites, particularly to the pulmonary parenchyma. The association of high FMRP expression with indicators of aggressive breast cancer disease and metastasis support a potential role for FMRP in breast tumor progression.

### Example 2

### Effect of FMRP Expression on Tumorigenicity

To investigate whether FMRP expression functionally affects tumorigenicity, invasiveness or metastasis formation, the highly metastatic murine breast cancer cells 4T1 (Tao et al., 2008) and TS/A (Nanni et al., 1983) were orthotopically injected into the mammary fat pad. The 4T1 mouse mammary tumor cell line leads, after orthotopic injection in the mammary fat pad, to a rapid and efficient metastatization of the target organs and results in an excellent mouse model for the study of metastatic progression of breast cancer in humans (Tao et al., 2008). The TS/A cell line (Nanni et al., 1983) was used for an independent experiment since it expresses lower levels of FMRP.

The resulting primary tumor showed higher levels of FMRP compared to normal breast tissues (FIG. 5 and data not shown) indicating that the mouse model replicates the findings in cancer patients. Interestingly, primary tumors derived from 4T1 cells formed a significantly higher number of lung metastases compared to TS/A cells (FIG. 6A; p<0.001, Student's t-test), which correlates with higher FMRP expression in 4T1 (FIG. 7A). The effect of FMRP knock-down was evaluated by means of shRNA lentivirus transduction in both cell lines. After Fmr1 silencing (FIG. 7C, E; FIG. 7B for choice of the shRNAs), the four cancer cell lines (4T1 and TS/A CTR shRNA and Fmr1 shRNA) were orthotopically implanted in the mammary fat pad of female mice with a syngenic background (Balb/c), and tumor growth was followed biweekly and analyzed after 29 and 35 days from the 4T1 and TS/A cell inoculation, respectively (FIG. 7D, F). Fmr1 silenced and control tumor cells grew (TS/A or 4T1) at a comparable rate and showed comparable tumor size and weight (FIG. 7D, F). Consistent herewith, in vitro cell growth was comparable between Fmr1 shRNA and CTR shRNA tumor cells (FIG. 8a-d).

Since in human primary tumors FMRP expression was correlated with prognostic indicators of lung metastasis, we investigated the development of lung metastases in mice injected with CTR shRNA and Fmr1 shRNA cells. Artificial reduction of FMRP levels by lentiviral transduction of shRNAs strikingly decreased the number of lung metastases formed by both cell lines by 50%, compared to mice injected with control cells transduced with a scrambled shRNA (n=13, P<0.05 and n=12, P<0.01, respectively) (FIGS. 6, B and C). Tumors generated with Fmr1-silenced and control cells showed comparable size (FIGS. 7, D and F) and weight (data not shown), indicating that the effect on number of metastases is indeed FMRP-dependent and not based on tumour dimensions (FIG. 6F). Poorly metastatic cells are believed to extravasate less compared to highly metastatic cells. Ability of CTR shRNA and Fmr1 shRNA TS/A cells to extravasate was tested by intravenous injection. Importantly, FMRP-silenced cells cause a reduced number of metastasis (FIG. 6F, p<0.05, Student's t-test) confirming and extending the findings obtained after orthotopic injection. Taken together, these results suggest that FMRP knockdown does not significantly affect tumor growth but clearly indicate a role for FMRP in cell invasiveness and metastasis formation.

### Example 3

### Expression of FMRP in Mouse Tumor Models

The expression level of FMRP was analysed in primary tumors (n=6) generated by orthotopic injection of CTR shRNA and Fmr1 shRNA 4T1 and TS/A cells into mammary fat pad. Importantly, as observed in human tumors, murine breast cancer cells showed high level of FMRP expression compared to normal breast tissue (FIG. 5, FIG. 9A, B and data not shown). FMRP was virtually absent in tumors generated by injection of Fmr1 shRNA cells (not shown). Breast cancer tissue generated by Fmr1 shRNA cells injection showed extensive necrosis with few or no apparently viable elements (not shown). The necrotic tissue dysplasia appears to be more evident in tumors generated with cells silenced for Fmr1 compared to control cells (not shown). Lung metastases generated in mice injected with control cells showed high level of FMRP expression (data not shown) while the few metastases generated by the Fmr1 shRNA cells showed a markedly reduced level of FMRP (data not shown). The large necrotic areas observed in tumors generated after orthotopic injection of Fmr1 shRNA TS/A cells lead us to further investigate cell proliferation in those tumor tissues, counting the number of cells expressing PCNA (see Methods). Strikingly, in the absence of FMRP, tumor cell proliferation is reduced (FIG. 9c, Fmr1 shRNA vs CTR p<0.05) while the percentage of necrotic areas (see Methods) is significantly higher compared to tumors generated by control cells (FIG. 9d, CTR shRNA vs Fmr1 shRNA p<0.01, Student's t-test). Recently, an effect of FMRP on proliferation of neuronal progenitor cell has been reported (Luo et al., 2010). These data are also in agreement with the significant correlation of high FMRP expression with Ki-67 observed in human primary tumors (FIG. 1b). While this opposite effect (decreased proliferation and increased necrosis in absence of FMRP) seems not to interfere with the final tumor growth (FIG. 7D, F and FIG. 8) it may have an enormous effect on metastasis generation.

These data, in addition to extend our findings in human to a mouse model, contribute to define a system to study the cellular and molecular events involving FMRP in metastasis formation.

### Example 4

### FMRP Regulates Adhesion and Cytoskeleton Remodeling Molecules during Tumor Progression and Metastasis Formation

The ability of cancer cells to disseminate from primary tumors and form metastasis is highly dependent on tumor cell invasion into the basal membrane, remodeling of the cytoskeleton, migration and adhesion (Nguyen et al., 2009). Indeed, during this transition cells change their shape and adhesive repertoire (Yilmaz and Christofori, 2009). To begin the metastatic cascade, cancer cells within the primary tumor acquire an invasive phenotype so that they can spread to distant sites. It is well known that loss of or low levels of expression of E-cadherin are a major hallmark of epithelial-mesenchymal transition (EMT) and cancer progression. Reduced E-cadherin expression promotes the shedding of the cancerous cells from the primary tumor (Schmalhofer et al., 2009; Yilmaz and Christofori, 2009), an important step of metastasis (Thiery et al., 2009) making E-cadherin a tumor suppressor protein (Kang et al., 2004; Berx and Van Roy, 2009). We therefore investigated the cell-cell adhesion property of the tumor cells. Ca²⁺-deprived control cells detached more easily from neighbouring cells and showed a more rounded shape than cells silenced for FMRP (FIG. 6D). Furthermore, in the absence of FMRP, cells kept their adhesion through protrusions (see arrows in FIG. 6D). To quantify cadherin-dependent adhesive properties, Fmr1 knocked-down 4T1 cells were plated on E-cadherin substrate resulting in an increased adhesion when compared to control cells (FIG. 6E, n=3, P<0.01). Adhesion to a substrate reflects the cell interaction with the matrix mimicking the tumor environment and it is correlated to the ability to metastasize (Akiyama et al., 1995).

In the nervous system, FMRP binds mRNAs encoding proteins involved in adhesion such as LI-cadherin (Miyashiro et al., 2003), Amyloid Precursor Protein (APP) (Napoli et al., 2008; Westmark and Matter, 2007), Neuroligin (Dahlhaus et al., 2010) and Adenomatous Polyposis Coli (APC) (Liao et al., 2008). Based on the above findings, we expected a dysregulation of cell adhesion molecules, especially E-cadherin, also in the tumors. Indeed, we found an inverse correlation between FMRP and endogenous E-cadherin in primary tumors generated after orthotopic injection of 4T1 cells (Fmr1 and CTR shRNA) (FIG. 10A, n=13, P<0.001 and FIG. 11) pointing to an FMRP-mediated regulation of E-cadherin. To extend these findings to human, nonmetastatic and metastatic breast cancer tissues (n=18; FIG. 4C) were stained for FMRP and E-cadherin. Low FMRP levels and high E-cadherin levels were detected in non-metastatic breast cancers, while the opposite was observed in metastatic breast cancers (FIG. 10B, n=18, P<0.05). Similarly, E-cadherin levels in FXS patients' lymphoblastoid cell lines (Primerano et al., 2002) correlate with the genotype, with more severe FMR1 mutations that express less FMRP having more E-cadherin (FIG. 12). Interestingly, correlation with vimentin is the opposite (FIG. 12). These data indicate that FMRP inhibits E-cadherin expression in human tumors and patient cell lines as previously shown in the mouse model. Finally, E-cadherin surface levels were also increased in Fmr1 shRNA 4T1 cells compared to control cells (FIG. 10C, n=3, P<0.01).

To investigate if the regulation is direct, we analysed E-cadherin mRNA binding to FMRP, using FMRP immunoprecipitation from 4T1 cell extracts (FIG. 4A, upper panel) coupled to RT-PCR of the co-precipitating mRNAs. We detected E-cadherin mRNA associated to the FMRP complex (FIG. 13A, lower panel; n=3, P<0.001 and see also FIG. 14A). The specificity of this interaction is shown by the much lower precipitation efficiency of Histone H3.3 and αTubulin mRNAs (FIG. 13A). Also in tumor tissue, E-cadherin was found associated with FMRP (FIG. 14B).

FMRP can regulate mRNA expression by inhibition of translation, or by modulating mRNA stability (Bassell and Warren, 2008; Bagni et al., 2005). Although vimentin mRNA levels were reduced significantly in Fmr1-silenced 4T1 cells, indicating an effect on stability (FIG. 15), RT-qPCR did not detect any changes in the steady state of the E-cadherin mRNA in control and Fmr1-silenced 4T1 cells (FIG. 15). Therefore, we analyzed the translational efficiency (polysome-mRNP distribution (Zalfa et al., 2003)) of E-cadherin mRNA in control and Fmr1-silenced cells. Indeed, translation of E-cadherin increased upon silencing of FMRP (FIG. 13B). In conclusion, FMRP binds to E-cadherin mRNA and downregulates its translation.

These data point towards a possible role of FMRP as inhibitor of E-cadherin mRNA translation, which may contribute to the induction of EMT in cancer cells thus increasing their metastatic potential. Without being bound to a particular mechanism, we propose that high FMRP expression would favour EMT through the rearrangement of the apico-basal polarity regulating Vimentin and E-cadherin expression and enhancing local invasion. Once in the bloodstream, tumor cells expressing high level of FMRP, would adhere and penetrate the basal membrane leading to an efficient spread of tumor cells possibly via FMRP regulation of integrins. Finally, cells would colonize the organ, due to a strong interaction with fibronectin leading to metastasis formation. Absence of FMRP would protect/delay these three crucial steps of cancer dissemination and metastatization.

To test the relevance of this hypothesis in vivo, mice were injected orthotopically with 4T1 CTR or Fmr1 shRNA cell lines carrying the GFP gene (and tested for FMRP expression, FIG. 16). After 5 weeks, blood GFP RNA levels were measured to quantify the number of tumour cells circulating in the bloodstream. Significantly less metastasizing tumour cells were observed in mice injected with Fmr1-silenced tumours (FIG. 16), indicating the validity of FMRP inhibition to prevent or reduce metastasis.

The molecular mechanism and the factors leading to the development of primary breast cancer and its progression are not fully elucidated. Loss or low expression level of E-cadherin function is a major hallmark of EMT and metastatisation making E-cadherin a tumor suppressor protein (Kang and Massagué, 2004). Both transcriptional and translational regulations usually cooperate for a cell efficient repression of E-cadherin (Yilmaz and Christofori, 2009; Kowalski et al., 2003) during metastatization although in few cases E-cadherin has also been detected in distal metastasis (Kowalski et al., 2003) indicating the complexity and variability of E-cadherin regulation. Metastatic progression possible involve quantitative rather than qualitative alteration of key-metastasis associated genes, as observed in the general upregulation of the translation enhancer eIF4E (Graff and Zimmer, 2003), present also in the FMRP complex (Napoli et al., 2008).

The most widely studied function of FMRP is role as repressor of mRNA translation in vivo (Bassell and Warren, 2008; Bagni et al., 2005) specifically acting in concert with the cap binding protein eIF4E, a general translational regulator which enhances translation of metastasis-related mRNAs (Graff and Zimmer, 2003), and with specific eIF4E-BPs (Napoli et al., 2008), emerging as promising therapeutic options for the treatment of cancer (Cencic et al., 2011; Malina et al., 2011). Metastatic progression is therefore enhanced by both qualitative and quantitative alterations of key metastasis-associated genes (Kang et al., 2004; Graff and Zimmer, 2003). Here, we show that FMR1 mRNA encoding for the Fragile X Mental Retardation Protein is significantly up-regulated in lymph node negative breast tumors that metastasize to the lung. In agreement, a reduction of FMRP levels in breast tumor cells decreases their ability to form lung metastases in mice. Furthermore, FMRP downregulates E-cadherin levels, important for cell adhesive properties and consequently tumor behaviour. It is conceivable that, in presence of high levels of FMRP, the reduced E-cadherin expression may contribute to the metastatic phenotype, evidence further supported by a significant correlation of FMRP with prognostic indicators of cancer dissemination.

Conclusion

The Fragile X Mental Retardation Protein (FMRP) is an RNA-binding protein involved in multiple steps of RNA metabolism in neurons, and is lacking or mutated in patients with the Fragile X Syndrome (FXS), the most frequent form of inherited mental retardation. In brain, FMRP regulates key mRNAs involved in cytoskeleton and spine remodelling. Herein, the first evidence is provided of high expression levels of FMRP in human primary breast cancers and distant metastases as well as a significant correlation between FMRP and prognostic indicators of aggressive breast cancer, development of lung metastasis, and disease recurrence. Reduction of FMRP in murine tumor cells decreases their ability to form pulmonary colonies. Finally, we identified translation of E-cadherin mRNA as a mechanism controlled by FMRP that affects tumor cell-adhesion properties.

Overall, these findings support a potential role for FMRP in breast tumor progression and dissemination and open major avenues into anticancer therapy aiming to down-regulate FMRP and/or pathways relevant for metastatisation in adults. Furthermore, they correlate a neurological disorder such as mental retardation with cancer progression showing that the same molecules and pathways are affected in two different diseases.

Material and Methods

Tissues from Patients.

Patients from the European Institute of Oncology (Milan, Italy, see FIGS. 2A, B and 4A).

Some of the specimens used in FIG. 1 were provided by the European Institute of Oncology (IEO, Milan, Italy). All human tissues were collected following standardized procedures and informed consent was obtained for all specimens linked with clinical data. Tissues used in FIG. 1 derived from the Molecular Pathology Unit at IFOM-IEO, according to procedures approved by the Institutional Ethical Board of the European Institute of Oncology.

Each sample was histopathologically evaluated to ensure the presence of at least 80% of tumor cells. The medical records of all patients were examined to obtain clinical and histopathological information. The histopathological diagnoses of the tumors were described according to the World Health Organization (WHO) International Classification of Disease for Oncology. The clinical staging was determined by the TNM Staging System. The malignancy of infiltrating carcinomas was scored according to the Scarff-Bloom-Richardson classification.

Patients from the University Hospital Leuven (Belgium, see FIG. 4B).

Some of the specimens used in FIG. 1 were provided by the University Hospital Leuven and samples collected according to a standardized method. Histopathologic examination was performed on hematoxylin and eosinstained sections and evaluated to ensure the presence of at least 80% of tumor cells. Tumors were classified and graded according to the WHO Classification and the Elston and Ellis grading system, respectively.

Patients from the IRCCS Hospital “Casa Sollievo della Sofferenza” (San Giovanni Rotondo, Italy, see FIG. 4C).

Specimens used in FIG. 10 were provided by the Laboratory of Oncology, IRCCS H. “Casa Sollievo della Sofferenza”, San Giovanni Rotondo, FG (Italy). Biopsy samples of in situ and invasive ductal breast carcinoma (n=18) were used for FMRP and Ecadherin immunohistochemistry (IHC). Immediately after surgery, the tumor samples were frozen at −80° C. Each sample was histopathologically evaluated to ensure the presence of at least 80% of tumor cells. The histopathological diagnoses of the tumors were described according to WHO International Classification of Disease for Oncology. The clinical stage was determined by the TNM Staging System.

Study of the FXS and Cancer Cohorts

A genetic register for fragile X has existed since 1985 at Central Manchester Foundation Trust (England). Individuals found to carry pre-mutation or full mutation on molecular testing are offered follow up through the register service. Unaffected obligate carriers are identified through testing cousins in different branches of the family. 226 female carriers with a definite molecular evidence of gene involvement were identified. Vital status was confirmed from the genetic register notes and from cancer registry data. Pre-mutation (decrease FMRP) and affected full mutation (absence of FMRP) patients were checked against the regional cancer register for all individuals. Relative risks for cancer were derived using a life table method and regional cancer incidence data from England.

Microarray and Statistical analyses

Affymetrix Microarray data and relative clinical and pathological information were downloaded from GEO (Gene expression Omnibus, http://www.ncbi.nlm.nih.gov/geo/) using the accession number GSE7390 for the TRANSBIG dataset, GSE2034 for the ERASMUS dataset, GSE2603 for the MSK-99 dataset, and NKI-295 at http://www.rii.com/publications/2002/nejm.html. Data were normalized using the MAS5.0 and processed in GeneSpring 7.3 (Agilent). Statistical analyses were performed on log2 median centered data using JMP IN 5.1 (SAS).

Human breast cancer samples stained with E-cadherin and FMRP were evaluated under code by three independent observers using a light microscope without the knowledge of either clinical or histological diagnosis. For each slide, a minimum of 10 fields was examined at 40× magnification. The percentage of positive cells/total cells, was evaluated for PCNA, FMRP and E-cadherin staining. The presence of tumor necrosis was verified by identifying karyorrhectic, pyknotic, or ghostlike tumor cells with loss of nuclear details. For each slide, a minimum of 10 fields was examined at 10X magnification. The score was determined by dividing the measured necrotic area by the total area of the field. The unpaired t test was used. Statistical significance was set at p<0.05.

Mice and Animal Care

Animal care was conducted conforming to the institutional guidelines that are in compliance with Italian laws (DL N116, GU, suppl 40, 18-2-1992), international laws and policies (European Community Council Directive 86/609, OJa L 358, 1, Dec. 12, 1987; National Institutes of Health Guide for the Care and Use of Laboratory Animals, US National Research Council, 1996) and approved by the Institutional Ethical Board at the Katholieke Universiteit of Leuven, Belgium. Nine weeks old Balb/c female mice were used in this study.

Immunohistochemical Analysis of FMRP on Human Tissue Microarrays (TMA)

Normal and tumor samples, formalin-fixed and paraffin-embedded (FFPE), were provided by the Pathology Departments of Ospedale Maggiore (Novara, Italy), Presidio Ospedaliero (Vimercate, Italy), Ospedale San Paolo (Milan, Italy) and Ospedale Sacco (Milano) were processed for the analysis of FMRP expression on multi-tumor TMAs. In colorectal, lung, larynx, prostate, and bladder tumors (T), the normal (N) samples were derived from the same patients whenever possible. In breast tumors, the normal samples were fibroadenomas and only in few cases paired normal counterparts (as specified in FIG. 2A). Samples were arrayed in four different TMAs (details in FIG. 2A), prepared essentially as previously described (Capra et al., 2006; Kononen et al., 1998). Briefly, two representative normal and tumor areas (diameter 0.6 mm) from each sample, previously identified on hematoxylineosin-stained sections, were removed from the donor blocks and deposited on the recipient block using a custom-built precision instrument (Tissue Arrayer-Beecher Instruments). For the FMRP study in human breast cancer and correlation analysis with clinically relevant parameters, we used data from a cohort of breast cancer patients enrolled in a surgical trial conducted at the European Institute of Oncology (EIO) between March 1998 and December 1999 (Veronesi et al., 2003) involving 441 consecutive patients (median age 55.8, range 37-75 years) with small-size primary breast cancers (pT1 and pT2<3.0 cm in diameter) and a mean follow-up period of 65 months. The overall clinical and pathological characteristics are given in FIG. 4A (and previously described) (Confalonieri et al., 2009). Immunohistochemical analysis of FMRP was performed by tissue microarray. Formalinfixed, paraffin-embedded tumor blocks were retrieved from the Pathology Department of the EIO and arrayed on different TMAs. For each patient, 2 representative cores were arrayed. Sections of 2 μm thickness of the TMA block were cut, mounted on glass slides and processed for IHC.

Estrogen- and progesterone-receptors, Ki67 and ErbB2 or HER2/neu, evaluated by IHC on whole tissue sections, were retrieved from histopathologic reports. ErbB2 or HER2/neu overexpression was evaluated according to the FDA-approved scoring system recommended by the DAKO Hercep Test. FMRP IHC was performed using polyclonal antibodies (Ferrari et al., 2007) (1:500 dilution) followed by detection with the EnVision Plus/HRP detection system (DAKO). A semi-quantitative approach was used to evaluate FMRP protein expression, scored as follows: 0, negative staining; 1, weak; 2, moderate; 3, intense. The samples displaying IHC scores >1.0 were considered positive, whereas those with scores ≦1.0 were considered negative. FMRP expression was positive at least in 80-90% of the tumor tissue analysed (in both samples from UZ Leuven and IFOM Milan).

Assessment of FMRP expression (for FIG. 1A): IHC signal was associated with the normal and tumor cell component and not with the adjacent or infiltrating stroma. Those cores in which the epithelial component was absent were discarded from the analysis. For the normal tissue counterparts we considered: for breast, normal-appearing ductal structures in fibroadenomas and breast tissues flanking tumour; for colon, glandular epithelium; for bladder, transitional epithelium. In normal colon tissue, the only two positive cases for FMRP expression showed diffuse morphological hyperplastic changes.

TMA IHC data were analysed using JMP IN 5.0 software (SAS). A P value of less than 0.05 was considered as significant.

Immunohistochemical Analysis on Human and Mouse Tumors

Human breast biopsy frozen sections were subjected to high temperature antigen retrieval in 1× Target Retrieval Solution (DAKO). The same protocol was applied to formalin-fixed and paraffin embedded murine breast tumors and brain after they were deparaffinised and rehydrated. Endogenous peroxidase activity was blocked by 3% H₂O₂. Subsequently, sections were incubated in 5% normal goat serum for FMRP or normal rabbit serum for E-cadherin. Mouse E-cadherin antibodies (1:200, BD Pharmigen), rabbit affinity purified FMRP antibodies (Ferrari et al., 2007) (1:50) and affinity purified IgG from pre-immune serum, were used overnight at 4° C. Biotinylated goat anti-rabbit or rabbit anti-mouse were used. Samples were then incubated with the avidin-biotin or ABC peroxidase complexes (Vector Laboratiories). The immunoreaction product was revealed using aminoethylcarbazole (AEC) or 3-3′ diaminobenzidine (DAB) as chromogenic substrates in presence of H₂O₂ (Biogenex). Control stainings were always performed omitting the primary antibody. Sections were counterstained in Mayer's acid hemalum or Harris counterstaining and analysed. Human and mouse breast cancer samples were blindly evaluated by three independent observers using a light microscope without knowing either the clinical or histological diagnosis. For each slice, a minimum of 10 fields was examined at 40× magnification. The Student's t-test was used. Statistical significance was set at P<0.05.

The invasive front was analysed as follows: The tumor ‘front’ was defined as the leading edge of the expanding infiltration into the stroma. The pattern of infiltration of the tumor edge is indeed variable and two different situations were observed:

1) Well-circumscribed pushing margin which abuts on and distorts the surrounding normal stroma, resulting in a smooth, rounded interface between the tumor and the surrounding tissue. In these instances there is often an associated dense lymphatic infiltrate at the margin.

2) Tumor that infiltrates the surrounding tissue diffusely enveloping pre-existing normal structures in its path, resulting in an irregular infiltrative margin.

Tumor and Lymphoblastoid Cell Lines

4T1 and TS/A cells (CTR and Fmr1 shRNA) were grown in DMEM-F12 media (Invitrogen) supplemented with Fetal Bovine Serum 10% (FBS, Invitrogen) and 1% penicillin-streptomycin (Invitrogen). Lymphoblastoid cell lines were cultured under the same media conditions but in suspension. All cells were kept at 37° C. in 5% CO₂.

Orthotopic Injection (0.1.) in Mice:

Lentivirus infected cells (a combination of two independent Fmr1 shRNAs 3/4 and a scrambled shRNA, CTR) were grown at 37° C. and 5% CO₂, washed in D-PBS (Invitrogen) and trypsinised. A small aliquot (dilution 1:1) was stained with trypan blue and counted to monitor cell viability. 3×10⁵ TS/A and 1×10⁶ 4T1 cells resuspended in 30 μl D-PBS were injected in the right second thoracic mammary fat pad. Tumor volume was measured with a caliper biweekly and calculated with the following formula π/6(r·l²), where l is the minor tumor axis and r the major tumor axis. 29 (4T1) or 35 (TS/A) days after injection, mice were sacrificed and lung metastases were stained with Indian ink and counted under a dissection microscope. Tumors were then divided in 4 parts and stored in liquid nitrogen for protein and RNA analysis, fixed in formalin and embedded in paraffin (FFPE) and kept at −80° C. in OCT.

Cell Growth

In vitro proliferation assay was measured by seeding 4000 cells/well for 10% FBS and 10.000 cells/well for 1% FBS into 48 well plate. Cells were allowed to adhere overnight at 10% FBS. After incubation, fresh medium containing 1% FBS was changed when appropriate. Cells were trypsinized and counted at 1 to 6 days by trypan blue staining (in triplicate for each well).

EDTA Adhesion Assay

4T1 CTR and Fmr1 shRNA cells were plated on plastic and grown to 80% confluence for 48 hr. Next, 0.5 mM EDTA was added in order to chelate calcium, and changes in the morphology of the cells were recorded by live imaging for 18 min using In Cell Analyzer workstation.

E-Cadherin-Dependent Adhesion Assay

A suspension of 50 μl containing 4×10⁵ cells ml-1 4T1 CTR and Fmr1 shRNA, were plated on a E-cadherin (R&D) coated 96 well plate and let to adhere for 20 min at 37° C. in a CO₂ incubator. The plate was shacked at 1500 rpm for 10-15 seconds and cells fixed with 4% PFA, washed and stained for 10 min with crystal violet (5 mg ml-1 in 2% Ethanol from Sigma-Aldrich). Cells were incubated with 2% SDS for 30 min at RT and optical reading of the plate was performed at 550 nm. Experiments were performed in triplicates with independent batches of cells.

Biotinylation of Cell Surface Proteins

4T1 CTR and Fmr1 shRNA cells were washed in cold PBS and incubated with and w/o Sulfo-NHS-LC-Biotin (Thermo Scientific) 0.2 mg/ml in PBS for 30 min at 4° C.

Excess biotin was quenched with three washes using 200 mM glycine. Cells where then lysed in STEN buffer and immunoprecipitated with streptavidin Dynabeads (Invitrogen) for 1 h at 4° C. Samples were then separated by SDS-PAGE.

E-cadherin surface levels were quantified using AIDA Image Analyzer v.4.22 and normalised for the input.

Western Blot

Cells and tumors were lysed in 100 mM NaCl, 10 mM MgCl₂, 10 mM Tris-HCl pH 7.5, 1% Triton X-100, 1 mM DTT, 40 U ml⁻¹ RNAse OUT (Invitrogen), 5 mM (β-glycerophosphate, 0.5 mM Na₃VO₄, 10 μl ml⁻¹ Protease inhibitor cocktail (PIC, Sigma) or 50 mM Tris HCl pH7,4, 150 mM NaCl, 1%, DOC, 1% NP-40, 10 μl ml⁻¹ PIC. After 5 min of incubation on ice, the lysates were centrifuged 15 min at 16,000 g at 4° C. 10-20 μg of supernatant were separated by SDSPAGE electrophoresis and transferred to a PVDF membrane (Millipore). Membranes were incubated using specific antibodies for FMRP (Ferrari et al., 2007) (1:1000), mouse Vinculin (1:2000, Sigma-Aldrich), mouse E-cadherin (1:500, BD Bioscience), mouse GAPDH (1:20000, Chemicon), rabbit rpS6 (1:1000, Cell Signaling) and signal was detected using an enhanced chemiluminescence kit (GE Healthcare). Membranes were then stained with Coomassie.

Immunoprecipitation Followed by RT-PCR and RT-qPCR

Cells were lysed in 100 or 250 mM NaCl, 50 mM Tris-HCl pH 7.4, 1% Triton X-100, 40 U ml⁻¹ RNAse OUT (Invitrogen), 10 mg ml⁻¹ PIC (Sigma). FMRP IP was performed using specific antibodies or purified rabbit IgGs as negative control and Protein A Dynabeads (Invitrogen). After incubation with the extracts, stringent washes with urea or low salt were performed. Lysis of mouse breast tumors was carried out on ice in 100 mM NaCl, 10 mM MgCl₂, 10 mM Tris-HCl pH 7.5, 1% Triton X-100, 1 mM DTT, 400 U ml⁻¹ RNAse OUT (Invitrogen), 10 μl ml⁻¹ PIC (Sigma), 5 mM β-glycerophosphate, 0.5 mM Na₃VO₄. After 5 min of incubation on ice, lysates were centrifuged for 5 min at 12,000 g at 4° C. and 500 μg of the supernatant was used for the IP. FMRP IP was performed using specific FMRP antibodies (Ferrari et al., 2007) or purified rabbit IgGs as negative control and Dynabeads Protein A immunoprecipitation kit (Invitrogen).

First-strand synthesis was performed using p(dN)6 and 100 U of M-MLV RTase (Invitrogen). RT-PCR was performed as previously described (Zalfa et al., 2007) using specific oligonucleotides to amplify Histone H3.3, Ferritin, Cytochrome C, αTubulin (negative control mRNAs) and E-cadherin mRNAs using the following oligos:

| E-cadherin (PCR product = 282 bp): | |
|---|---|
| (SEQ ID NO: 1) | |
| fw: | 5′-GCCGGAGAGGCACCTGGAGA-3′; |
| (SEQ ID NO: 2) | |
| rev: | 5′-TTCAGAGGCAGGGTCGCG-3′ |
| Vimentin: | |
| (SEQ ID NO: 3) | |
| fw: | 5′-TATGTGACCCGGTCCTCGG-3′; |
| (SEQ ID NO: 4) | |
| rev: | 5′-AGGTTGTCCCGCTCCACCT-3′ |
| Histone H3.3 (PCR product = 569 bp): | |
| (SEQ ID NO: 5) | |
| fw: | 5′-GCGGCCCGGAAAAGCGCGCCCTCTACT-3′; |
| (SEQ ID NO: 6) | |
| rev | 5′-GTATCACCCATCCCTTCTGCATATTA-3′ |
| Ferritin (PCR product = 340 bp): | |
| (SEQ ID NO: 7) | |
| fw | 5′-TTCGTCGTTCGCCCGCTCCA-3′; |
| (SEQ ID NO: 8) | |
| rev | 5′-GCCCGCTCTCCCAGTCATCA-3′ |
| Cytochrome C (PCR product = 370 bp): | |
| (SEQ ID NO: 9) | |
| fw | 5′-GGTTACTTCGCGGAGTGG-3′; |
| (SEQ ID NO: 10) | |
| rev | 5′-GCCATTTGGTGGGGCACC-3′ |
| αTubulin (PCR product = 600 bp): | |
| (SEQ ID NO: 11) | |
| fw | 5′-TTTTCCACAGCTTTGGTGGGGG-3′; |
| (SEQ ID NO: 12) | |
| rev | 5′-TCTTGATGGTGGCAATGGCAG-3′ |

Real Time PCR (RT-PCR) was performed using an ABI 7300 Sequence Detector with dual-labeled TaqMan probes (Applied Biosystems). Mouse Histone H3.3, E-cadherin (Cdh1), and αTubulin mRNAs were detected with Pre-Developed TaqMan gene expression assays Mm00787223_s1, Mm01247357_m1, Mm00502040_m1 respectively. Relative immunoprecipitation efficiencies, normalized to control IgGs, were calculated as follows: 2^{-[ΔCt(specific IgGs)-ΔCt(control IgGs)]}=2^{-ΔΔCt}, where ΔCt equals Ct(IP)−Ct(Input). TaqMan Universal PCR Master Mix (ABI 4304437) was used. Cycles: 2 min at 50° C., 10 min at 95° C., followed by 40 cycles of 15 sec at 95° C. and 1 min at 60°.

Polysomes-mRNP Analysis

Tumor or metastatic 4T1 cells were homogenized in 10 mM Tris-HCl pH 7.5, 100 mM NaCl, 10 mM MgCl₂, 1% Triton-X100, 1 mM dithiothreitol DTT, 40 u/mL RNasin supplemented with 100 mg/ml cycloheximide. After 5 min of incubation on ice, the extract was centrifuged for 5 min at 12,000 g at 4° C. The supernatant was loaded onto a 15-50% (w/v) sucrose gradient and sedimented by centrifugation at 4° C. for 110 min at 37,000 rpm in a Beckman SW41 rotor (Fullerton).

Each gradient was collected into 10 fractions followed by the addition of 1% SDS (final concentration), 40 pg of hexogen (in vitro transcribed) BC200 RNAs, 10 μg glycogen, and proteinase K (100 μg/ml) and incubated for 30 min 37° C. The hexogen BC200 RNA was used to monitor possible RNA loss during RNA phenol/chloroform extraction and precipitation from each fraction. RNAs were precipitated with 0.2M NaOAc and 0.7 vol of isopropanol. The pellets were then resuspended in 30 μl of ddH2O. The RNA fractions 1-5 (polysomal fraction, P) and 6-10 (mRNP fraction, NP) were pooled and RNA quality/quantity was assessed by 1.8% agarose formaldehyde gel electrophoresis and spectrophotometry (ND-1000 spectrophotometer, Nanodrop Technology). mRNAs of interest (Histone H3.3 and E-cadherin mRNAs) were quantified by RT-qPCR (see above), and the translational efficiency was calculated as follows: 2^{-[ΔCt(P)-ΔCt(NP)]}=2^{-ΔΔCt}, where ΔCt equals Ct(Histone H3.3 or E-cadherin mRNAs)−Ct(BC200 RNA).

Silencing of Fmr1 Using Lentiviral Vector

To silence Fmr1 mRNA, five independent shRNAs plasmids (# SHCLND-NM_{—}008031 Sigma-Aldrich) were tested.

1) shRNA #1: targeting Fmr1 3′UTR
(SEQ ID NO: 13)
5′-CCGGCCTTACATAAACATCAGCTTACTCGAGTAAGCTGATGTTTAT
GTAAGGTTTTTG-3′
2) shRNA #2: targeting Fmr1 CDS
(SEQ ID NO: 14)
5′-CCGGCGCACCAAGTTGTCTCTTATACTCGAGTATAAGAGACAACTT
GGTGCGTTTTTG-3′
3) shRNA #3: targeting Fmr1 CDS
(SEQ ID NO: 15)
5′-CCGGCCACGAAACTTAGTAGGCAAACTCGAGTTTGCCTACTAAGTT
TCGTGGTTTTTG-3′
4) shRNA #4: targeting Fmr1 CDS
(SEQ ID NO: 16)
5′- CCGGCCACCACCAAATCGTACAGATCTCGAGATCTGTACGATTTG
GTGGTGGTTTTTG-3′
5) shRNA #5: targeting Fmr1 CDS
(SEQ ID NO: 17)
5′- CCGGGAGGATGATAAAGGGTGAGTTCTCGAGAACTCACCCTTTAT
CATCCTCTTTTTG-3′
6) Control scramble shRNA (#SHC002 Sigma-Aldrich)
(SEQ ID NO: 18)
5′-CCGGCAACAAGATGAAGAGCACCAACTCGAGTTGGTGCTCTTCATC
TTGTTGTTTTT-3′

shRNA plasmids were transiently transfected (48 hrs) in 4T1 cells using lipofectamine (FIG. 7B). Only shRNAs #3, #4, #5 were then used to generate lentiviral particles to silence FMRP in tumour cells.

Second generation plasmids (Naldini et al., 1996) were used to generate transduction particles using HEK293T as packaging cells by calcium phosphate transfection method (Chen and Okayama, 1987). Virus-containing supernatants from HEK293T cells were collected after 24 hr incubation and filtered through 0.22 μm filters (Millipore). Combination of two different viruses carrying different shRNAs (3/4, 4/5, 3/5) was added to 70% confluent cells (4T1 or TS/A) in presence of 8 μg ml-1 polybrene (Sigma) and incubated overnight. Infection efficiency was checked by parallel infection with GFP lentivirus. The parental vector (pLKO.1-puro) (Sigma-Aldrich) allows monitoring stable transfection via puromycin resistance selection. The virus has been propagated in episomal form and kept at −80° C. The silencing efficiency was verified by Western blot analysis (FIGS. 7C and E). The most efficient combination (Fmr1 shRNA 3/4) and the control shRNA (Sigma) were used.

### REFERENCES

Adereth, Y., Dammai, V., Kose, N., Li, R., & Hsu, T., RNA-dependent integrin alpha3 protein localization regulated by the Muscleblind-like protein MLP1. Nat Cell Biol 7 (12), 1240-1247 (2005).

Agulhon, C. et al., Expression of FMR1, FXR1, and FXR2 genes in human prenatal tissues. J Neuropathol Exp Neurol 58 (8), 867-880 (1999).

Akiyama S K, Olden K, Yamada K M. Fibronectin and integrins in invasion and metastasis. Cancer Metastasis Rev. 1995; 14(3):173-89.

Ashley C T, Sutcliffe J S, Kunst C B, Leiner H A, Eichler E E, Nelson D L, Warren S T. Human and murine FMR-1: alternative splicing and translational initiation downstream of the CGG-repeat. Nat Genet. 1993; 4(3):244-51.

Bagni, C. & Greenough, W. T., From mRNP trafficking to spine dysmorphogenesis: the roots of fragile X syndrome. Nat. Rev. Neurosci. 6 (5), 376-387 (2005).

Barbano R, Copetti M, Perrone G, Pazienza V, Muscarella L A, Balsamo T, Storlazzi C T, Ripoli M, Rinaldi M, Valori V M, Latiano T P, Maiello E, Stanziale P, Carella M, Mangia A, Pellegrini F, Bisceglia M, Muda A O, Altomare V, Murgo R, Fazio V M, Parrella P. High RAD51 mRNA expression characterize estrogen receptor-positive/progesteron receptor-negative breast cancer and is associated with patient's outcome. Int J Cancer. 2011; 129(3):536-45.

Bassell, G. J. & Warren, S. T., Fragile X syndrome: loss of local mRNA regulation alters synaptic development and function. Neuron 60 (2), 201-214 (2008).

Berx G, van Roy F. Involvement of members of the cadherin superfamily in cancer. Cold Spring Harb Perspect Biol. 2009; 1(6):a003129.

Bilousova, T. V. et al., Minocycline promotes dendritic spine maturation and improves behavioural performance in the fragile X mouse model. J Med Genet 46 (2), 94-102 (2009).

Brown V, Jin P, Ceman S, Darnell J C, O′Donnell W T, Tenenbaum S A, Jin X, Feng Y, Wilkinson K D, Keene J D, Darnell R B, Warren S T. Microarray identification of FMRP-associated brain mRNAs and altered mRNA translational profiles in fragile X syndrome. Cell. 2001; 107(4):477-87.

Capra M, Nuciforo P G, Confalonieri S, Quarto M, Bianchi M, Nebuloni M, Boldorini R, Pallotti F, Viale G, Gishizky M L, Draetta G F, Di Fiore P P. Frequent alterations in the expression of serine/threonine kinases in human cancers. Cancer Res. 2006; 66(16):8147-54.

Cencic R, Hall D R, Robert F, Du Y, Min J, Li L, Qui M, Lewis I, Kurtkaya S, Dingledine R, Fu H, Kozakov D, Vajda S, Pelletier J. Reversing chemoresistance by small molecule inhibition of the translation initiation complex eIF4F. Proc Natl Acad Sci USA. 2011; 108(3):1046-51.

Centonze, D. et al., Abnormal striatal GABA transmission in the mouse model for the fragile X syndrome. Biol Psychiatry 63 (10), 963-973 (2008).

Chaffer C L, Weinberg R A. A perspective on cancer cell metastasis. Science. 2011; 331(6024):1559-64.

Chen C, Okayama H. High-efficiency transformation of mammalian cells by plasmid DNA. Mol Cell Biol. 1987; 7(8):2745-52.

Chen, W., Heierhorst, J., Brosius, J., & Tiedge, H., Expression of neural BC1 RNA: induction in murine tumours. Eur J Cancer 33 (2), 288-292 (1997).

Chiurazzi, P., Neri, G., & Oostra, B. A., Understanding the biological underpinnings of fragile X syndrome. Curr. Opin. Pediatr. 15 (6), 559-566 (2003).

Confalonieri S, Quarto M, Goisis G, Nuciforo P, Donzelli M, Jodice G, Pelosi G, Viale G, Pece S, Di Fiore P P. Alterations of ubiquitin ligases in human cancer and their association with the natural history of the tumor. Oncogene. 2009; 28(33):2959-68.

Dahlhaus R, El-Husseini A. Altered neuroligin expression is involved in social deficits in a mouse model of the fragile X syndrome. Behav Brain Res. 2010; 208(1):96-105.

De Rubeis, S. & Bagni, C., Fragile X mental retardation protein control of neuronal mRNA metabolism: Insights into mRNA stability. Mol Cell Neurosci 43 (1), 43-50 (2009).

Desmedt C, Piette F, Loi S, Wang Y, Lallemand F, Haibe-Kains B, Viale G, Delorenzi M, Zhang Y, d′Assignies M S, Bergh J, Lidereau R, Ellis P, Harris A L, Klijn J G, Foekens J A, Cardoso F, Piccart M J, Buyse M, Sotiriou C; TRANSBIG Consortium. Strong time dependence of the 76-gene prognostic signature for node-negative breast cancer patients in the TRANSBIG multicenter independent validation series. Clin Cancer Res. 2007; 13(11):3207-14.

Dictenberg, J. B., Swanger, S. A., Antar, L. N., Singer, R. H., & Bassell, G. J., A direct role for FMRP in activity-dependent dendritic mRNA transport links filopodial-spine morphogenesis to fragile X syndrome. Dev Cell 14 (6), 926-939 (2008).

Elston C W, Ellis I O. Pathological prognostic factors in breast cancer. I. The value of histological grade in breast cancer: experience from a large study with long-term follow-up. Histopathology 1991; 19; 403-410.

Ferrari, F. et al., The fragile X mental retardation protein-RNP granules show an mGluR-dependent localization in the post-synaptic spines. Mol Cell Neurosci 34 (3), 343-354 (2007).

Fidler I J. The pathogenesis of cancer metastasis: the ‘seed and soil’ hypothesis revisited. Nat Rev Cancer. 2003; 3(6):453-8.

Fitzgibbons P L, Page D L, Weaver D, Thor A D, Allred D C, Clark G M, Ruby S G, O′Malley F, Simpson J F, Connolly J L, Hayes D F, Edge S B, Lichter A, Schnitt S J. Prognostic factors in breast cancer. College of American Pathologists Consensus Statement 1999. Arch Pathol Lab Med. 2000; 124(7):966-78.

Fulchignoni-Lataud, M. C., Olchwang, S., Serre, J. L. The fragile X CGG repeat shows a marked level of instability in hereditary non-polyposis colorectal cancer patients. Europ. J. Hum. Genet. 5: 89-93 (1997)

Goldhirsch A, Glick J H, Gelber R D, Coates A S, Senn H J. Meeting highlights: International Consensus Panel on the Treatment of Primary Breast Cancer. Seventh International Conference on Adjuvant Therapy of Primary Breast Cancer. J Clin Oncol. 2001; 19(18):3817-27.

Graff, J. R. & Zimmer, S. G., Translational control and metastatic progression: enhanced activity of the mRNA cap-binding protein eIF-4E selectively enhances translation of metastasis-related mRNAs. Clin Exp Metastasis 20 (3), 265-273 (2003).

Hood, J. D. & Cheresh, D. A., Role of integrins in cell invasion and migration. Nat Rev Cancer 2 (2), 91-100 (2002).

Iacoangeli, A. et al., BC200 RNA in invasive and preinvasive breast cancer. Carcinogenesis 25 (11), 2125-2133 (2004).

Kalkunte, R., Macarthur, D., & Morton, R., Glioblastoma in a boy with fragile X: an unusual case of neuroprotection. Arch Dis Child 92 (9), 795-796 (2007).

Kang Y, Massagué J. Epithelial-mesenchymal transitions: twist in development and metastasis. Cell. 2004; 118(3):277-9.

Kim, T. Y., Vigil, D., Der, C. J., & Juliano, R. L., Role of DLC-1, a tumor suppressor protein with RhoGAP activity, in regulation of the cytoskeleton and cell motility. Cancer Metastasis Rev 28 (1-2), 77-83 (2009).

Kononen J, Bubendorf L, Kallioniemi A, Bärlund M, Schraml P, Leighton S, Torhorst J, Mihatsch M J, Sauter G, Kallioniemi O P. Tissue microarrays for high-throughput molecular profiling of tumor specimens. Nat Med. 1998; 4(7):844-7.

Kowalski, P. J., Rubin, M. A., & Kleer, C. G., E-cadherin expression in primary carcinomas of the breast and its distant metastases. Breast Cancer Res 5 (6), R217-222 (2003).

Kunda, P., Craig, G., Dominguez, V., & Baum, B., Abi, Sra1, and Kette control the stability and localization of SCAR/WAVE to regulate the formation of actin-based protrusions. Curr Biol 13 (21), 1867-1875 (2003).

Liao L, Park S K, Xu T, Vanderklish P, Yates J R 3rd. Quantitative proteomic analysis of primary neurons reveals diverse changes in synaptic protein content in fmr1 knockout mice. Proc Natl Acad Sci USA. 2008; 105(40):15281-6.

Lu, R. et al., The fragile X protein controls microtubule-associated protein 1B translation and microtubule stability in brain neuron development. Proc. Natl. Acad. Sci. USA 101 (42), 15201-15206 (2004).

Luo Y, Shan G, Guo W, Smrt R D, Johnson E B, Li X, Pfeiffer R L, Szulwach K E, Duan R, Barkho B Z, Li W, Liu C, Jin P, Zhao X. Fragile x mental retardation protein regulates proliferation and differentiation of adult neural stem/progenitor cells. PLoS Genet. 2010; 6(4):e1000898.

Macara, I. G. & Mili, S., Polarity and differential inheritance—universal attributes of life? Cell 135 (5), 801-812 (2008).

Malina A, Cencic R, Pelletier J. Targeting translation dependence in cancer. Oncotarget. 2011; 2(1-2):76-88.

Micalizzi D S, Ford H L. Epithelial-mesenchymal transition in development and cancer. Future Oncol. 2009; 5(8):1129-43.

Mili, S., Moissoglu, K., & Macara, I. G., Genome-wide screen reveals APC-associated RNAs enriched in cell protrusions. Nature 453 (7191), 115-119 (2008).

Minn A J, Gupta G P, Siegel P M, Bos P D, Shu W, Giri D D, Viale A, Olshen A B, Gerald W L, Massagué J. Genes that mediate breast cancer metastasis to lung. Nature. 2005; 436(7050):518-24.

Miyashiro, K. Y. et al., RNA cargoes associating with FMRP reveal deficits in cellular functioning in Fmr1 null mice. Neuron 37 (3), 417-431 (2003).

Naldini L, Blömer U, Gallay P, Ory D, Mulligan R, Gage F H, Verma I M, Trono D. In vivo gene delivery and stable transduction of nondividing cells by a lentiviral vector. Science. 1996; 272(5259):263-7.

Nanni, P., de Giovanni, C., Lollini, P. L., Nicoletti, G., & Prodi, G., TS/A: a new metastasizing cell line from a BALB/c spontaneous mammary adenocarcinoma. Clin Exp Metastasis 1 (4), 373-380 (1983).

Napoli, I. et al., The fragile X syndrome protein represses activity-dependent translation through CYFIP1, a new 4E-BP. Cell 134 (6), 1042-1054 (2008).

Nguyen, D. X., Bos, P. D., & Massague, J., Metastasis: from dissemination to organ-specific colonization. Nat Rev Cancer 9 (4), 274-284 (2009).

Olson, M. F. & Sahai, E., The actin cytoskeleton in cancer cell motility. Clin Exp Metastasis 26 (4), 273-287 (2009).

Onder, T. T. et al., Loss of E-cadherin promotes metastasis via multiple downstream transcriptional pathways. Cancer Res 68 (10), 3645-3654 (2008).

Oostra B A, Chiurazzi P. The fragile X gene and its function. Clin Genet. 2001; 60(6):399-408.

Park, S. et al., Elongation factor 2 and fragile X mental retardation protein control the dynamic translation of Arc/Arg3.1 essential for mGluR-LTD. Neuron 59 (1), 70-83 (2008).

Polyak K, Weinberg R A. Transitions between epithelial and mesenchymal states: acquisition of malignant and stem cell traits. Nat Rev Cancer. 2009; 9(4):265-73.

Primerano B, Tassone F, Hagerman R J, Hagerman P, Amaldi F, Bagni C. Reduced FMR1 mRNA translation efficiency in fragile X patients with premutations. RNA. 2002; 8(12):1482-8.

Sahai, E., Illuminating the metastatic process. Nat Rev Cancer 7 (10), 737-749 (2007).

Schenck, A. et al., CYFIP/Sra-1 controls neuronal connectivity in Drosophila and links the Rac1 GTPase pathway to the fragile X protein. Neuron 38 (6), 887-898 (2003).

Schmalhofer O, Brabletz S, Brabletz T. E-cadherin, beta-catenin, and ZEB1 in malignant progression of cancer. Cancer Metastasis Rev. 2009; 28(1-2):151-66.

Schmid, R. S. & Maness, P. F., L1 and NCAM adhesion molecules as signaling coreceptors in neuronal migration and process outgrowth. Curr Opin Neurobiol 18 (3), 245-250 (2008).

Schultz-Pedersen, S., Hasle, H., Olsen, J. H., & Friedrich, U., Evidence of decreased risk of cancer in individuals with fragile X. Am J Med Genet 103 (3), 226-230 (2001).

Silva, J. M. et al., Cyfip1 is a putative invasion suppressor in epithelial cancers. Cell 137 (6), 1047-1061 (2009).

Tao, K., Fang, M., Alroy, J., & Sahagian, G. G., Imagable 4T1 model for the study of late stage breast cancer. BMC Cancer 8, 228 (2008).

Thiery J P, Acloque H, Huang R Y, Nieto M A. Epithelial-mesenchymal transitions in development and disease. Cell. 2009; 139(5):871-90.

van de Vijver M J, He Y D, van't Veer L J, Dai H, Hart A A, Voskuil D W, Schreiber G J, Peterse J L, Roberts C, Marton M J, Parrish M, Atsma D, Witteveen A, Glas A, Delahaye L, van der Velde T, Bartelink H, Rodenhuis S, Rutgers E T, Friend S H, Bernards R. A gene-expression signature as a predictor of survival in breast cancer. N Engl J Med. 2002; 347(25):1999-2009.

Verheij C, de Graaff E, Bakker C E, Willemsen R, Willems P J, Meijer N, Galjaard H, Reuser A J, Oostra B A, Hoogeveen A T. Characterization of FMR1 proteins isolated from different tissues. Hum Mol Genet. 1995; 4(5):895-901.

Verkerk A J, de Graaff E, De Boulle K, Eichler E E, Konecki D S, Reyniers E, Manca A, Poustka A, Willems P J, Nelson D L, et al. Alternative splicing in the fragile X gene FMR1. Hum Mol Genet. 1993; 2(4):399-404.

Veronesi U, Paganelli G, Viale G, Luini A, Zurrida S, Galimberti V, Intra M, Veronesi P, Robertson C, Maisonneuve P, Renne G, De Cicco C, De Lucia F, Gennari R. A randomized comparison of sentinel-node biopsy with routine axillary dissection in breast cancer. N Engl J Med. 2003; 349(6):546-53.

Wang Y, Klijn J G, Zhang Y, Sieuwerts A M, Look M P, Yang F, Talantov D, Timmermans M, Meijer-van Gelder M E, Yu J, Jatkoe T, Berns E M, Atkins D, Foekens J A. Gene-expression profiles to predict distant metastasis of lymph-node-negative primary breast cancer. Lancet. 2005; 365(9460):671-9.

Westmark C J, Matter J S. FMRP mediates mGluR5-dependent translation of amyloid precursor protein. PLoS Biol. 2007; 5(3):e52.

Yilmaz, M. & Christofori, G., EMT, the cytoskeleton, and cancer cell invasion. Cancer Metastasis Rev 28 (1-2), 15-33 (2009).

Zalfa, F. et al., The fragile X syndrome protein FMRP associates with BC1 RNA and regulates the translation of specific mRNAs at synapses. Cell 112 (3), 317-327 (2003).

Zalfa, F. et al., A new function for the fragile X mental retardation protein in regulation of PSD-95 mRNA stability. Nat Neurosci 10 (5), 578-587 (2007).

1. A method of assessing metastatic potential of a tumor in a subject, the method comprising:
determining levels of FMR1 gene product in said tumor by analyzing a sample of the tumor,
thereafter assessing the tumor's metastatic potential based upon the determined levels of FMR1 gene product, and
altering therapy of the subject based upon the assessment of the tumor's metastatic potential.

2. The method of claim 1, further comprising:
correlating increased levels of FMR1 gene product to an increased risk of metastasis.

3. The method of claim 1, wherein the FMR1 gene product is Fmr1 mRNA.

4. The method of claim 1, wherein the FMR1 gene product is FMRP protein.

5. The method of claim 1, wherein the tumor is selected from the group consisting of breast cancer, colon cancer, and bladder cancer tumors.

6. The method of claim 5, wherein the tumor is lymph node negative breast cancer tumor.

7. The method of claim 1, wherein the levels are determined in vitro.

8. A method of inhibiting metastasis of a tumor in a subject, the method comprising:
determining that the subject has a tumor, and
administering an FMR1 inhibitor to the subject
so as to inhibit metastasis of the tumor in the subject.

9. The method of claim 8, wherein the FMR1 inhibitor is an inhibitor of Fmr1 mRNA.

10. The method of claim 8, wherein the FMR1 inhibitor is an inhibitor of FMRP protein.

11. The method of claim 8, wherein the tumor is breast cancer tumor.

12. The method of claim 11, wherein the breast cancer tumor is lymph node negative breast cancer tumor.

13. A pharmaceutical composition comprising an amount of an FMR1 inhibitor sufficient to inhibit FMR1 in a subject after being administered thereto.

14. (canceled)

15. The method of claim 9, wherein the FMR1 inhibitor is an siRNA specific for Fmr1.

16. The method of claim 10, wherein the FMR1 inhibitor is an anti-FMRP antibody.

17. A method of treating a subject determined to have a tumor, the method comprising:
administering an amount of FMR1 inhibitor to the subject effective to inhibit metastasis of the tumor, and
monitoring the tumor for metastasis.

18. The method of claim 8, wherein the FMR1 inhibitor is an siRNA specific for Fmr1 or an anti-FMRP antibody.

19. The method of claim 18, wherein the tumor is a breast cancer tumor.

20. The method of claim 19, wherein the breast cancer tumor is a lymph node negative breast cancer tumor.

21. The method of claim 8, further comprising:
determining levels of FMR1 gene product in the tumor by analyzing a sample of the tumor,
thereafter assessing the tumor's metastatic potential based upon the determined levels of FMR1 gene product, and
altering therapy of the subject based upon the assessment of the tumor's metastatic potential.


SEQUENCE LISTING (18 sequences)

SEQ ID NO: 1 - DNA, 20 bp, Artificial Sequence
  Primer
gccggagaggcacctggaga

SEQ ID NO: 2 - DNA, 18 bp, Artificial Sequence
  Primer
ttcagaggcagggtcgcg

SEQ ID NO: 3 - DNA, 19 bp, Artificial Sequence
  Primer
tatgtgacccggtcctcgg

SEQ ID NO: 4 - DNA, 19 bp, Artificial Sequence
  Primer
aggttgtcccgctccacct

SEQ ID NO: 5 - DNA, 27 bp, Artificial Sequence
  Primer
gcggcccggaaaagcgcgccctctact

SEQ ID NO: 6 - DNA, 26 bp, Artificial Sequence
  Primer
gtatcacccatcccttctgcatatta

SEQ ID NO: 7 - DNA, 20 bp, Artificial Sequence
  Primer
ttcgtcgttcgcccgctcca

SEQ ID NO: 8 - DNA, 20 bp, Artificial Sequence
  Primer
gcccgctctcccagtcatca

SEQ ID NO: 9 - DNA, 18 bp, Artificial Sequence
  Primer
ggttacttcgcggagtgg

SEQ ID NO: 10 - DNA, 18 bp, Artificial Sequence
  Primer
gccatttggtggggcacc

SEQ ID NO: 11 - DNA, 22 bp, Artificial Sequence
  Primer
ttttccacagctttggtggggg

SEQ ID NO: 12 - DNA, 21 bp, Artificial Sequence
  Primer
tcttgatggtggcaatggcag

SEQ ID NO: 13 - DNA, 58 bp, Artificial Sequence
  Primer
ccggccttacataaacatcagcttactcgagtaagctgatgtttatgtaaggtttttg

SEQ ID NO: 14 - DNA, 58 bp, Artificial Sequence
  Primer
ccggcgcaccaagttgtctcttatactcgagtataagagacaacttggtgcgtttttg

SEQ ID NO: 15 - DNA, 58 bp, Artificial Sequence
  Primer
ccggccacgaaacttagtaggcaaactcgagtttgcctactaagtttcgtggtttttg

SEQ ID NO: 16 - DNA, 58 bp, Artificial Sequence
  Primer
ccggccaccaccaaatcgtacagatctcgagatctgtacgatttggtggtggtttttg

SEQ ID NO: 17 - DNA, 58 bp, Artificial Sequence
  Primer
ccgggaggatgataaagggtgagttctcgagaactcaccctttatcatcctctttttg

SEQ ID NO: 18 - DNA, 57 bp, Artificial Sequence
  Primer
ccggcaacaagatgaagagcaccaactcgagttggtgctcttcatcttgttgttttt